(19) United States
(12) Patent Application Publication
(10) Pub. No.: US 2024/0240816 A1
(43) Pub. Date: Jul. 18, 2024

(54) SYSTEM FOR FLUID SEPARATION, METHODS OF MAKING AND USING THE SAME

(71) Applicant: DeCarbon Air, LLC, Mount Joy, PA (US)

(72) Inventor: Dustin Matthew EPLEE, Mount Joy, PA (US)

(73) Assignee: DeCarbon Air, LLC, Mount Joy, PA (US)

(21) Appl. No.: 18/411,809

(22) Filed: Jan. 12, 2024

Related U.S. Application Data
(60) Provisional application No. 63/480,093, filed on Jan. 16, 2023.

(51) Int. Cl. F24F 8/20; A61L 9/16

(52) U.S. Cl. CPC F24F 8/20; A61L 9/16; A61L 2209/16

(57) ABSTRACT

A device for separation of fluid species is disclosed. The device comprises at least one header connected to a monoblock. The header transitions one or more fluid streams between bulk flow and multi-channel flow patterns using a transition element. The header is attached to a monoblock, which conditions an untreated process fluid stream by adjusting the temperature of, and/or separating at least a portion of one or more fluid species from, the untreated process fluid stream. Fluid separation is accomplished by the use of high boiling point liquids infused into the pore structure of the monoblock with the outputs being a conditioned process fluid stream and an exhaust fluid stream. Methods of making the device and methods of using the device to separate at least a portion of fluid species from a process fluid stream are also disclosed.

### CROSS-REFERENCE TO RELATED APPLICATION

This application claims the benefit of priority to U.S. Provisional Application Ser. No. 63/480,093, filed on Jan. 16, 2023, the contents of which are incorporated herein by reference in its entirety.

### TECHNICAL FIELD

The present disclosure generally relates to the field of fluid separation. The present disclosure also relates to materials, devices, and apparatus for performing the same. The present disclosure further relates to methods of using such devices and apparatus in industrial processes, indoor air cleaning, heating, ventilation, and air conditioning (“HVAC”) systems, and other fields.

### BACKGROUND

Indoor air quality (“IAQ”) can be a material factor for maintaining a healthy and comfortable living or working environment in certain conditions. Various IAQ factors are measured and maintained, and are often tailored to the specific needs of the particular building, whether it be a house, an office building, or a warehouse. Indoor air problems arise from a broad variety of pollutants, and depending on the particular application, may include particulate matter (“PM”), formaldehyde, volatile organic compounds (“VOCs”), carbon dioxide (“CO2”), semi-volatile organic compounds, house dust mites, mold, bacteria, and associated health effects such as sick building syndrome symptoms, asthma, allergies, Legionnaires' disease, lung cancer, and airborne infections, such as SARS and COVID. IAQ has traditionally been addressed in part by introduction of outdoor air into buildings. For example, outdoor air has been introduced to buildings to reduce the concentration of PM, VOCs, and CO2, or to control indoor humidity levels.

The present disclosure was inspired in part by both the global coronavirus disease 2019 (COVID-19) outbreak and an increase in outdoor pollution levels, leading the inventor to question the industry-wide assumption that introduction of outdoor air is the best way to control IAQ factors in building air.

When used to treat building air, the innovation behind the present disclosure can minimize and potentially eliminate the need to introduce outdoor air to buildings while maintaining the IAQ factors within levels that are comfortable and in compliance with relevant IAQ standards.

In 2022, the American National Standards Institute (ANSI) and the American Society of Heating, Refrigerating and Air-Conditioning Engineers (ASHRAE) amended their standard for ventilation and acceptable indoor air quality. See ANSI/ASHRAE Standard 62.1-2022: Ventilation and Acceptable Indoor Air Quality, ASHRAE (September 2022) (hereinafter “the Standard”). The ANSI/ASHRAE Standard 62.1 is a recognized standard for ventilation system design and acceptable IAQ. The Standard specifies minimum ventilation rates and other measures in order to minimize adverse health effects for occupants. See Standards 62.1 & 62.2, ASHRAE (September 2022), https://www.ashrae.org/technical-resources/bookstore/standards-62-1-62-2. The 2022 edition of Standard 62.1 reflects how IAQ goes beyond ventilation requirements. The Standard provides a procedure for determining the amount of outdoor air required for concentrations of certain compounds and particulate matter that are 2.5 μm or less in diameter (“PM2.5”) in the indoor environment to comply with the Standard's design limits. See Standard 62.1-2022 at 15, 25-26. The Standard's design limits for various compounds and PM2.5, along with citation to their relevant authorities, are reproduced below:

TABLE 1

| | | Design | |
|---|---|---|---|
| Compound or PM2.5 | Cognizant Authority | Limit | |
| Acetaldehyde | Cal EPA CREL (June 2016) | 140 | μg/m³ |
| Acetone | AgBB LCI | 1,200 | μg/m³ |
| Benzene | Cal EPA CREL (June 2016) | 3 | μg/m³ |
| Dichloromethane | Cal EPA CREL (June 2016) | 400 | μg/m³ |
| Formaldehyde | Cal EPA 8-hour CREL (2004) | 33 | μg/m³ |
| Naphthalene | Cal EPA CREL (June 2016) | 9 | μg/m³ |
| Phenol | AgBB LCI | 10 | μg/m³ |
| Tetrachloroethylene | Cal EPA CREL (June 2016) | 35 | μg/m³ |
| Toluene | Cal EPA CREL (June 2016) | 300 | μg/m³ |
| 1,1,1-trichloroethane | Cal EPA CREL (June 2016) | 1000 | μg/m³ |
| Xylene, total | AgBB LCI | 500 | μg/m³ |
| Carbon monoxide | U.S. EPA NAAQS | 9 | ppm |
| PM2.5 | U.S. EPA NAAQS | 12 | μg/m³ |
| | (annual mean) | | |
| Ozone | U.S. EPA NAAQS | 70 | ppb |
| Ammonia | Cal EPA CREL (June 2016) | 200 | μg/m³ |

Some embodiments of the present disclosure enable reduction of the concentration of these compounds within the Standard's design limits without dilution from outdoor air.

Fluid separation technologies are important in various industries, as explained in further detail below. Gas separation technology also contributes to environmental initiatives, notably in carbon capture. Furthermore, gas separation aids in improving IAQ by selectively removing contaminants and regulating air composition. Traditional and current fluid separation technologies suffer from substantial downfalls, including requiring substantial energy input, complex mechanisms, high expense, and frequent maintenance. The current separation techniques are discussed below.

Physical Absorption. This method involves passing gases through a liquid medium acting as a solvent. It encounters challenges in targeting particular gases with similar solubility characteristics, hampering precise separation. Energy-intensive desorption from the liquid absorbent impacts overall efficiency.

Chemical Reactions. Utilizing selective chemical reactions, this method forms compounds for gas separation. However, the intricacy of the processes, coupled with complex reaction mechanisms, leads to higher operational and maintenance costs. Scaling for industrial use presents challenges, reducing efficiency and cost-effectiveness.

Membrane Separation. This method relies on semi-permeable membranes, differentiating gases based on size, solubility, or diffusivity. Membranes face challenges such as degradation over time and fouling, reducing efficiency. Continuous processing becomes a concern due to these limitations.

Adsorption Processes. This method involves gas molecules adhering to solid surfaces (adsorbents) through cyclic variations of pressure or temperature. Challenges include saturation of the adsorbents, leading to reduced efficiency and energy-intensive regeneration. The cyclical nature complicates continuous processing.

Cryogenic Distillation. This technique involves liquefaction at temperatures below −150° C. to separate gases by boiling point using distillation columns. This technique demands high energy input, complex infrastructure, and the use of coolants with a high global warming potential.

The existing fluid separation technologies face significant challenges, emphasizing the need for advancements. An ideal technology should be robust, durable, resistant to fouling, energy-efficient, suitable for continuous processing, and environmentally friendly, depending on the application for the fluid separation technology.

In view of this long-felt need in the industry, the present technology addresses and overcomes one or more of the foregoing problems. Thus, the innovation behind the present disclosure can minimize and potentially eliminate the need to introduce outdoor air to buildings while maintaining the IAQ factors within levels that are comfortable and in compliance with relevant IAQ standards.

The disclosed monoblocks, materials, systems and methods are designed to maintain or improve IAQ factors, while overcoming one or more of the problems set forth above and/or other problems of the prior art, while also being modular and compatible with various applications and industries.

### SUMMARY

Consistent with some disclosed embodiments, a header configured to divide a bulk flow of fluid into a multi-channel flow of fluid is disclosed. The header may include at least one inlet, a transition element, a plurality of routing channels, and at least one outlet. In some embodiments, the first inlet is configured to receive a first bulk flow of fluid, and the first outlet comprises a plurality of openings that are fluidly connected to a first group of fluid flow channels. In some embodiments, a first group of fluid flow channels and a second group of fluid flow channels are on a device attached to the header. In some embodiments, the transition element is configured to divide the first bulk flow of fluid into a first multi-channel flow of fluid. In some embodiments, a plurality of first routing channels is configured to route the first multi-channel flow of fluid to a first outlet, which may be fluidly connected to a first group of fluid flow channels.

Consistent with some disclosed embodiments, an apparatus comprising a monoblock for fluid separation is disclosed. The monoblock device may have a plurality of fluid flow channels, including a first group of fluid flow channels, which is independent from a second group of fluid flow channels. In some embodiments, the fluid flow channels are separated by channel walls, which may be porous, selectively permeable, and formed of a material that the monoblock is formed of. In some embodiments, the channel walls are impregnated with one or more high boiling point liquids and at least a portion of the first group of fluid flow channels being adjacent to at least a portion of the second group of fluid flow channels.

Consistent with some disclosed embodiments a method of reducing the concentration of components of a fluid stream is disclosed. The method includes introducing a process fluid stream comprising a mixture of fluid species into a device. In some embodiments, the device may have (a) at least one header having a transition element, a plurality of routing channels, and at least one outlet. In some embodiments, a first inlet is configured to receive a first bulk flow of fluid, the first outlet comprises a plurality of openings that are fluidly connected to a first group of fluid flow channels, the transition element is configured to divide the first bulk flow of fluid into a multi-channel flow of fluid, and a plurality of first routing channels are configured to route the first multi-channel flow of fluid to a first outlet.

In some embodiments, the device may further have (b) a monoblock having a plurality of fluid flow channels including the first group of fluid flow channels and a second group of fluid flow channels. The plurality of fluid flow channels are separated by channel walls, the channel walls being porous, selectively permeable, and formed of a material that the monoblock is formed of, the channel walls being impregnated with one or more high boiling point liquids, and at least a portion of the first group of fluid flow channels being adjacent to at least a portion of the second group of fluid flow channels.

Consistent with some disclosed embodiments, a method of manufacturing a header is disclosed. In some embodiments, the method may comprise one or more of the following steps: (a) providing a monoblock formed of a porous material; the monoblock having a plurality of fluid flow channels formed therein the plurality of fluid flow channels being separated by channel walls; the channel walls being porous and selectively permeable; (b) providing a negative mold comprising fingers adapted to register in a subset of the plurality of fluid flow channels; (c) inserting the fingers of the negative mold into the subset of the plurality of fluid flow channels; (d) introducing a liquid polymeric material into the negative mold; (e) allowing said liquid polymeric material to cure; and (f) removing the negative mold to establish a header.

### BRIEF DESCRIPTION OF THE DRAWINGS

The accompanying drawings, which are incorporated in and constitute a part of this specification, illustrate disclosed embodiments and, together with the description, serve to explain the disclosed embodiments. The particulars shown are by way of example and for purposes of illustrative discussion of embodiments of the present disclosure. The description taken with the drawings makes apparent to those skilled in the art how embodiments of the present disclosure may be practiced.

FIG. 1A is a perspective view of the header structure consistent with some disclosed embodiments.

FIG. 1B is a detailed view of a portion of the header structure depicted in FIG. 1A.

FIG. 1C is a detailed view of the channelizer of the header structure depicted in FIG. 1A.

FIG. 1D is a detailed view of the channelizer depicted in FIG. 1C.

FIG. 2A is a perspective view of the header structure consistent with some disclosed embodiments.

FIG. 2B is an alternate perspective view of the header structure depicted in FIG. 2A.

FIG. 3A is a perspective view of the header structure consistent with some disclosed embodiments.

FIG. 3B is a detailed view of a portion of the header structure depicted in FIG. 3A.

FIG. 4A is a perspective view of the header structure consistent with some disclosed embodiments.

FIGS. 4B and 4C are detailed views of a portion of the header structure depicted in FIG. 4A.

FIG. 5 is a perspective view of the monoblock structure depicting the flow of fluid streams consistent with some disclosed embodiments.

FIGS. 6A to 6L are exemplary arrangements of the channels formed within a monoblock consistent with some disclosed embodiments, including FIG. 6A showing the channels arranged in a checkerboard pattern, FIG. 6B showing the channels arranged in an off-set checkerboard pattern, FIG. 6C showing the channels arranged in a honeycomb pattern, FIG. 6D showing the channels arranged in a diamond pattern, FIG. 6E showing the channels arranged in a diamond and triangle pattern, FIG. 6F showing the channels arranged in an alternate diamond and triangle pattern, FIG. 6G showing the channels arranged in an equilateral triangle pattern, FIG. 6H showing the channels arranged in an offset triangle pattern, FIG. 6I showing the channels arranged in a right triangle pattern, FIG. 6J showing the channels arranged in a triangle and pentagon pattern, FIG. 6K showing the channels arranged in an alternate triangle and pentagon pattern, and FIG. 6L showing the channels arranged in a triangle and hexagon pattern.

FIG. 7A is an exploded perspective view of the gas separation apparatus consistent with some disclosed embodiments.

FIG. 7B is a detailed view of the gas separation apparatus of FIG. 7A.

FIG. 8 depicts the channelizer routing fluid streams into and out of the monoblock consistent with some disclosed embodiments.

FIG. 9 is the mechanism of target fluid flow through the channel walls of a monoblock consistent with some disclosed embodiments.

FIG. 10 is a two-dimensional front view of the monoblock structure consistent with some disclosed embodiments.

FIG. 11 is a block diagram of two monoblocks arranged in series consistent with some disclosed embodiments.

FIG. 12A is a block diagram of a fluid conditioning unit (“FCU”) consistent with some disclosed embodiments.

FIG. 12B is a perspective view of a line replaceable unit (“LRU”), which is a portion of the FCU in FIG. 12A.

### DETAILED DESCRIPTION

### Definitions

As used herein, “monoblock” means a structure that is created in one piece, composed generally of a material that may be a homogeneous material or a complex compound with some variation in the composition of the material throughout the structure.

As used herein, “bulk flow” characterizes a fluid stream moving cohesively within a consolidated structure, such as a pipe or manifold, in contrast to a scenario where the fluid stream traverses through multiple distinct channels.

As used herein, “fluid” means a substance that flows and takes the shape of its container, encompassing both liquids and gases.

As used herein, “separation efficiency” means the percent change of a component fluid species between the process fluid stream and the conditioned process fluid stream. For example, if the process fluid stream has a CO2 concentration of 1.0 g/L and the conditioned process fluid stream has a CO2 concentration of 0.1 g/L, the separation efficiency is (1.0 g/L−0.1 g/L)/(1.0 g/L)=90%.

As used herein “process fluid stream” means a mixed stream of fluid species that is introduced to the monoblock.

As used herein, “conditioned process fluid stream” means the process fluid stream that was conditioned by the monoblock in terms of temperature and/or separation of at least a portion of one or more fluid species.

As used herein, “exhaust fluid stream” means the fluid stream exiting the monoblock comprising at least a portion of one or more fluid species separated from the process fluid stream.

As used herein, “multi-channel flow” characterizes a fluid stream moving within a pattern that involves the concurrent movement through multiple conduits. pipes.

As used herein, “header” means a structural component located adjacent to a second structure, intended to alter the flow of substances into the second structure.

As used herein, “structurally distinct” explains that a first structure is separate or dissimilar to a second structure in terms of material, function, position, or other attributes.

As used herein, “fluidly connected” indicates a connection between two structures that enables the movement of a fluid from the first structure into or through the second structure.

As used herein, “route,” when used as a verb, describes the action of guiding fluids from one location or configuration to another designated location or configuration.

As used herein, “porous” describes a property of a material or structure where spaces or openings enable the passage of fluids through the material or structure.

As used herein, “selectively permeable” describes the property of a permeable material or structure having some preference for certain fluids to pass through, based on any of the fluid's physical properties, chemical properties, or a combination thereof. The preference for certain fluids need not be a strong one, but merely a preference which is statistically significant.

As used herein, “high boiling point” indicates the quality of a liquid with notable resistance to boiling or vaporization, especially in conditions of elevated temperatures or low pressure. A “high boiling point” is established at a minimum of 100° C.

As used herein, “impregnated” indicates the incorporation of one material or substance into some structure. The word “impregnated” may be illustrated by a sponge being impregnated with water or an HVAC system being impregnated with air.

As used herein, “deactivate” means to make (something, typically a biological component or a virus) inactive by disconnecting, capturing, immobilizing, quarantining, destroying, or killing it.

As used herein, “pore size” refers to the size of a pore in terms of its dimensions when viewed into the pore: the width or diameter rather than the depth.

As used herein, “absorb” means the process of taking in, bonding to, adhering to, attracting to, connecting to, or soaking up a substance through either chemical or physical action.

As used herein, “negative mold” means a mold that has the inverse shape of the desired object or component, also known as female molds. A casting material is usually inserted into the negative mold to create the desired component.

As used herein, “hardened” means the condition of having become physically hard or tougher, evident through factors like a higher Rockwell hardness rating, an elevated modulus, or increased resistance to deformation.

As used herein, “introducing” means placing or bringing something into another entity.

As used herein, “allowing” means furnishing the necessary time or opportunity for a particular purpose.

As used herein, “subset” means any number, up to and including, a complete set.

As used herein, “plenum structure” means a space utilized as a pathway for the distribution of air, including a housing, a manifold, ductwork, a chamber, an air distribution box, and a cavity. For example, in HVAC systems a plenum is an enclosed space serving for air distribution or housing HVAC components and facilitates even distribution of conditioned air throughout a building.

As used herein, “decarbonize” means to reduce or eliminate hydrocarbon emissions from a process such as manufacturing or the production of energy, or in an environment such as a building or the outdoors.

The present disclosure is generally directed to a monoblock with a plurality of channels formed within it, wherein the channel walls are porous. The porous channel walls are impregnated with a high boiling point liquid, which selectively allows passage of a subset of fluid species from one channel, through the porous channel wall, and into an adjacent channel for removal as an exhaust fluid stream.

For example, if the process fluid stream for separation is an industrial flue gas and a target fluid is CO2, flue gas would enter through a first group of fluid flow channels in the monoblock. Then, at least a portion of the CO2 contained in the flue gas would be adsorbed by a high boiling point liquid, which is selected to have some affinity for CO2 and is impregnated in the channel walls. The CO2 absorbed by the high boiling point liquid would diffuse across the channel wall to an adjacent second group of fluid flow channels, where the CO2 would desorb from the high boiling point liquid and exit the monoblock as an exhaust fluid stream for downstream use or sequestration.

Thus, technology disclosed herein has the capacity to decarbonize industries including agriculture, buildings, transport, military submarines, and heavy industry to enable reductions in greenhouse emissions. The monoblock can also be paired with heated or cooled fluid steams to simultaneously adjust the temperature of, and separate unwanted fluid species from, an incoming fluid stream. The monoblock may also include a header fitted thereto, which routes either or both of a process fluid stream into the monoblock for fluid separation and an exhaust fluid stream out of the monoblock.

The monoblock for fluid separation and related systems and methods described herein are an improvement over existing commercially available products because it eliminates, at least the following: (1) the vapor compression cycle; (2) refrigerants with high global warming potential; (3) refrigerant compressors; (4) energy-intensive metals and associated corrosion; (5) condensate; (6) ice formation; and (7) dangerous charging/disposing of refrigerants.

The technology disclosed herein facilitates selective fluid separation while reducing the complexity to achieve that selective separation compared to the prior art. For example, the embodiments disclosed herein do not require the use of complex ancillary equipment other than a single pump to introduce fluid to, or remove fluid from, the monoblock. The disclosed embodiments are also highly modular and therefore scalable to the needs of the operation and can be easily built on-site, shifting the point of postponement. Various disclosed embodiments of the present invention are more robust, durable, resistant to fouling, energy efficient, amenable to continuous processing, and not reliant on materials with high global warming potential, thereby addressing many of the shortcomings of the current fluid separation techniques.

In some embodiments, the novel fluid separation apparatus, method, and system has a variety of uses in many fields including industrial processes, healthcare, environmental initiatives, indoor air cleaning, HVAC systems, oil and gas, recreational sports and activities, chemical and petrochemical, food and beverage industries, aerospace, laboratory and research, and water treatment.

In some embodiments, industrial processes that would benefit from this novel fluid separation apparatus, method, and system include the following: hydrogen and oxygen production by ionization of water and separating the resulting hydrogen and oxygen; separation of gases in the synthesis of ammonia, a key component in fertilizer production; producing oxygen by separating oxygen from air for use in combustion and oxyfuel systems; steel production, including purifying blast furnace gasses, enriching oxygen for enhancing the efficiency of basic oxygen steelmaking and improving combustion for faster steel production, and removing impurities for increased quality of the steel product; generating nitrogen for inerting applications, such as preventing explosions and controlling oxygen levels in tanks, contributing to industrial and environmental safety.

In some embodiments, electronics manufacturing processes that would benefit from this novel fluid separation apparatus, method, and system include the following: semiconductor manufacturing, including maintaining a controlled and pure environment for chemical vapor deposition (CVD) and physical vapor deposition (PVD) to prevent impurities from affecting the quality of semiconductor materials; electronics manufacturing, including producing inerting gasses like nitrogen and argon to prevent oxidation and improve the precision of electronic components; creating clean room environments for electronics manufacturing by removing particulates and contaminants from the air to produce sensitive electronic devices.

In some embodiments, polymer manufacturing processes that would benefit from this novel fluid separation apparatus, method, and system include the following: removing impurities, unreacted monomers, and undesired components from monomers to ensure the quality and purity of the monomers before polymerization; capturing byproduct gases for recycling or appropriate disposal; controlling the composition and purity of input gases used for foaming or extrusion processes.

In some embodiments, healthcare applications that would benefit from this novel fluid separation apparatus, method, and system include the following: producing medical-grade gases including oxidation, nitrogen, and nitrous oxide; concentrating oxygen gas for delivery to patients with respiratory conditions; producing anesthesia gases with precise concentrations and purity for safe and effective use of medical procedures; humidifying gases delivered to patients for respiratory care to prevent irritation; analyzing blood gases including oxygen and carbon dioxide; purifying gases used in the cryopreservation of biological materials; producing medical gases for hemodialysis processes; producing high purity gases for laboratory applications such as research, diagnostics, and medical testing; purifying gases used for medical imaging techniques such as: helium purification for magnetic resonance imaging; isotope separation for positron emission topography or single-photon emission computed tomography; purification of xenon gas used as a contrast agent in xenon-enhanced computed tomography imaging; concentration of the necessary gases for mapping brain activity with functional magnetic resonance imaging; and purification of hyper polarized noble gasses like helium-3 and the xenon-129 for hyperpolarized gas imaging.

In some embodiments, hyperbaric chambers would benefit from this novel fluid separation apparatus, method, and system by producing oxygen gas with precise concentrations and purity to deliver high-pressure oxygen in hyperbaric chambers for therapeutic purposes. Hyperbaric chambers are known to have beneficial medical effects such as wound and injury healing by increasing oxygen levels in tissues, stimulating the formation of new blood vessels (angiogenesis), and supporting the body's natural healing mechanisms. Hyperbaric oxygen therapy has anti-inflammatory, which is beneficial for many medical conditions. It also treats decompression sickness in divers and carbon monoxide poisoning by displacing the excess nitrogen and carbon monoxide, respectively, from the body. It also has antimicrobial properties, contributing to the control of certain infections by enhancing the body's immune response and promoting the activity of white blood cells. It has a positive impact on non-healing ulcers, such as diabetic foot ulcers, and helps oxygen get to the brain in ischemic strokes. It is being explored as a possible supportive measure for certain neurological conditions, including traumatic brain injuries and neurological rehabilitation, and for assisting cancer therapies. See generally, Hyperbaric Oxygen Therapy, Johns Hopkins Med., https://www.hopkinsmedicine.org/health/treatment-tests-and-therapies/hyperbaric-oxygen-therapy; Yafit Hachmo et al., Hyperbaric Oxygen Therapy Increases Telomere Length and Decreases Immunosenescence in Isolated Blood Cells: a Prospective Trial, 12 AGING 22445 (2020).

In some embodiments, environmental initiatives that would benefit from this novel fluid separation apparatus, method, and system include the following: separating and capturing carbon dioxide emissions from industrial processes; capturing NOx, SOx, mercury, VOCs, and other compounds from industrial processes; upgrading biogas produced from organic waste to enhance the quality of the biogas for use in energy production or injection into natural gas pipelines; capturing methane and other components from the gases emitted from landfills; purifying oxygen for liquid oxygen production, which is used in environmental remediation.

In some embodiments, indoor air cleaning applications that would benefit from this novel fluid separation apparatus, method, and system include the following: capturing harmful or unwanted gasses such as CO2, VOCs, odor-causing gases and vapors, and radon; purifying oxygen for supplementation in building air; Reducing concentration of oxygen from a building for medical reasons such as inducing sleep; controlling water vapor, and thus, humidity; deactivating pathogens, bacteria, and viruses; Monitoring specific gas species in building air; concentrating oxygen from outdoor air to enhance oxygen concentration indoors along with reduction of indoor pollutants can have substantial health benefits including increased sleep quality, mental performance, productivity, general well-being, exercise performance and recovery, altitude acclimatation, healing, cognitive function, reduced sensitivity to allergens, and potentially increased life expectancy. See generally, J. B. West., Oxygen Enrichment of Room Air to Improve Well-Being and Productivity at High Altitude, 5 Int'l J. Occupational and Env't Health 187 (1999); Jan Stepanek et al., Supplemental CO2 Improves Oxygen Saturation, Oxygen Tension, and Cerebral Oxygenation in Acutely Hypoxic Healthy Subjects, PHYSIOLOGICAL REP. (Jul. 29, 2020); Silvia Ulrich et al., Effect of Breathing Oxygen-Enriched Air on Exercise Performance in Patients With Precapillary Pulmonary Hypertension: Randomized, Sham-Controlled Cross-Over Trial, 38 Eur. Heart J. 1159 (2017); Robert J. Laumbach et al., Personal Interventions for Reducing Exposure and Risk for Outdoor Air Pollution: An Official American Thoracic Society Workshop Report, 18 Annals Am. Thoracic Soc'y 1435 (2021).

In some embodiments, the oil and gas industry would benefit from this novel fluid separation apparatus, method, and system in the following ways: sweetening natural gas by removing impurities like hydrogen sulfide to meet environmental and safety standards; dehydrating natural gas to prevent hydrate formation; separating and capturing methane, ethane, propane, and other components of natural gas for various applications; removing heavy hydrocarbons from natural gas streams, preventing issues such as fouling and ensuring the quality of the gas for further processing; extracting helium from natural gas sources in the oil and gas industry; desalting crude oil by removing water and salts from the oil-water mixture, ensuring the quality of crude oil for further refining; producing nitrogen gas for blanketing storage tanks, preventing explosions, and inerting processes.

In some embodiments, recreational sports and activities that would benefit from this novel fluid separation apparatus, method, and system include the following: rebreathers for scuba diving, where CO2 is separated from residual oxygen in the diver's exhalation, and oxygen is directed to the diver's inhalation; removing CO2 from inhaled air for snorkeling; concentrating oxygen for supplementation during high altitude activities such as paragliding, rock climbing, mountaineering, or skiing.

In some embodiments, chemical applications that would benefit from this novel fluid separation apparatus, method, and system include the following: generating nitrogen for blanketing flammable gasses or inerting processes; removing certain chemical reaction product gasses to drive chemical reaction processes.

In some embodiments, food and beverage industry applications that would benefit from this novel fluid separation apparatus, method, and system include the following: reducing oxygen or nitrogen blanketing for more effective packaging and preservation of food products; producing pure CO2 or nitrogen gas for carbonation of beverages; extracting CO2 in brewing processes for controlled carbonation of fermented products; removing oxygen from edible oil processing to prevent rancidity and extend the shelf life of oils; removing hydrogen sulfide from sugar production processes, to ensure quality and safety.

In some embodiments, aerospace applications that would benefit from this novel fluid separation apparatus, method, and system include the following: maintaining oxygen concentrations in high altitude aerospace applications; purifying oxygen for oxygen reserves; producing oxygen by ionization of water in space missions; producing high-purity oxygen for fuel cells in aircraft, enhancing the efficiency of fuel cell-powered systems and reducing emissions; removing CO2 from enclosed aerospace vehicles; generating nitrogen for aircraft tire inflation, enhancing safety and performance by preventing moisture-related issues in tires; Recovering and purifying hydrogen from rocket exhaust, allowing for reuse in propulsion systems and reducing waste; extracting carbon dioxide from spacesuits; providing high-purity oxygen for combustion in hypersonic vehicles, supporting efficient propulsion; producing oxygen-enriched air for high-altitude parachute jumps, ensuring the safety and well-being of parachutists at extreme altitudes.

In some embodiments, water treatment applications that would benefit from this novel fluid separation apparatus, method, and system include the following: removing dissolved CO2 for pH adjustment; removing dissolved oxygen from boiler feedwater, preventing corrosion and ensuring the longevity and efficiency of boiler systems; removing certain dissolved gases from drinking water such as methane, VOCs, or hydrogen sulfide, preventing potential safety hazards and improving the quality of drinking water; generating ozone for water disinfection and microbial control; removing ammonia from water to prevent algae-related issues.

### Monoblock Structure:

The basic structure of the monoblock is generally an extruded geometric shape, for example, a cuboid, cylinder, or hexoid. The monoblock has a front face with a plurality of cells that extend into the monoblock to form fluid flow channels. For example, in a cylindrical monoblock, the multiple cells would appear on the circular front face of the monoblock and would extend into the monoblock to form parallel fluid flow channels through a length of the cylinder. At least some of the fluid flow channels formed in the monoblock extend through the entirety of the monoblock, creating open cells on the rear face of the monoblock.

The monoblock includes a plurality of parallel fluid flow channels extending through a length of the monoblock in the direction of the extrusion. Among the plurality of parallel fluid flow channels, there are at least two independent groups of fluid flow channels, each with its own purposes.

The cells appearing on the front face of the monoblock can be a geometric shape or collection of shapes that are capable of a repeating pattern. For example, a checkerboard pattern, offset checkerboard pattern, honeycomb pattern, quatrefoil pattern, rectangle pattern, diamond pattern, triangle pattern, triangle and diamond pattern, triangle and rectangle pattern, or a combination thereof, so long as the geometric shapes share borders.

The monoblock structure may be modified from commercially available monoblocks. The dimensions of one such commercially available monoblock are about 100 mm×100 mm×300 mm, such as 50 mm×50 mm×150 mm. Commercially available cylindrical monoblocks may be up to 400 mm in diameter with varying depths.

The density of cells on the face of the monoblock may be, for example, 200 cells/in\hat{ }2 to 1200 cells/in\hat{ }2 such as, for example, 350 cells/in\hat{ }2 to 550 cells/in\hat{ }2. The ratio of open cells to the overall monoblock face may be, for example, 0.6 to 0.95, such as, for example, 0.75 to 0.95. The fluid flow channel length in the monoblock may be, for example, 50 mm to 500 mm, such as, for example, 150 mm to 300 mm. Channel wall density of about 0.30 to 0.50. The volume of the fluid flow channels may be, for example, 0.012 in\hat{ }3.

In some embodiments, and depending on the application for the monoblock, the channel wall thickness may be about 2 mil to 15 mil (0.002 in to 0.015 in), such as about 8 mil to 10 mil (0.008 in to 0.010 in) for low pressure drop and fast vapor transmission.

In designing monoblocks for fluid separation, there is a balance between geometric surface area and pressure drop. The pressure drop (“ΔP”) across the monoblock depends linearly on flow velocity and length.

ΔP=2*f* [symbol] *ρ*v\hat{ }2*Gc*Dh where f is the friction factor (dimensionless); Dh the hydraulic diameter (cm); Gc the gravitational constant; [symbol] the monoblock length (cm); v the velocity of the fluid flow through the channel (cm/s); and ρ the fluid density (g/cm\hat{ }3). This fundamental equation may assist a person of ordinary skill in the art to design monoblock geometric parameters such as cell density or wall thickness to meet the constraints of external processing requirements such as space velocity, flow rates, pressure drop, etc.

### Monoblock Material:

The monoblock is formed of an absorbent, high-surface area, and porous material. In certain embodiments, the monoblock material may also have thermal stability, mechanical strength, and chemical resistance. The material may be activated carbon, inorganic materials, clays, activated alumina, talc, or a combination thereof. The monoblock may be made of a mixture of binders, activated carbon, and a ceramic material. For example, cordierite is a ceramic material with commercial availability, thermal stability, and low thermal expansion. Depending on the application for the technology, the monoblock material may be chosen for properties including the ability to withstand operating temperatures of 260° C., pressure differentials of 14.7 psi, and/or prolonged exposure to caustic fluids.

The surface area of the material chosen for the monoblock may be, for example, about 200 m\hat{ }2/g to over 1000 m\hat{ }2/g and more preferentially for example about 500 m\hat{ }2/g to about 550 m\hat{ }2/g. The geometric surface area may be between about 20 in\hat{ }2/in\hat{ }3 and about 200 in\hat{ }2/in\hat{ }3. The pore volume of the material chosen for the monoblock may be, for example, about 0.20 mL/g to about 1.0 mL/g and more preferentially, for example, about 0.20 mL/g to about 0.30 mL/g.

The carbon source for an activated carbon monoblock material may be wood, peat, coal, coconut, lignite, petroleum pitch, petroleum coke, coal tar pitch, fruit pits, nut shells, sawdust, wood flour, synthetic polymer, polymer, or any other carbon source known to a person of ordinary skill in the art, or a combination thereof. The carbon can be activated by chemical process, thermal process, or any other process for activating carbon known to a person of ordinary skill in the art. Activating carbon increases its surface area and porosity.

Inorganic monoblock materials may include zeolites, porous silica, porous alumina, pillared clays, molecular sieves, and porous polymers.

In some embodiments, the monoblock material may have charged functional groups or specific charges on its surface to enhance or alter the function of the ionic liquid.

Examples of anions useful within the monoblock may include: [BF4] Tetrafluoroborate, [C(CN)3] Tricyanomethanide, [DCA] Dicyanamide, [Doc] bis(2-ethylhexyl)sulfosuccinate, [ESU] Ethyl sulfate, [Et2PO4] Diethylphosphate, [EtOAc] Ethoxyacetate, [FAP] Trifluoro phosphate, [Gly] Glycolate, [Inda] Indazolide, [Lac] L(+) lactate, [Lev] Levulinate, [Mal] Malonate, [Me2PO4] Trimethylphosphate, [PF6] Hexafluorophosphate, [Pho] Phenolate, [Pro] Proline, [Pro] Prolinate, [Tf2N]bis(trifluoromethylsulfonyl)imide, [TfA] Trifluoroacetate, [TfO] Trifluoromethane sulfonate, [Ac] Acetate, B(CN)4 Tetracyanoborate, BF4 Tetrafluoroborate, C(CN)3 Tricyanomethanide, DCA or N(CH)2 Dicyanamide, PF6 Hexafluorophosphate, RPO4 Alkyl phosphate, RSO4 Alkyl sulfate, Tf2N Bis(trifluoromethylsulfonyl)imide, TfO Trifluoromethylsulfonate, TFSI (=Tf2N) Bis(trifluoromethylsulfonyl)imide, or any other relevant negatively charged functional group known by a person of ordinary skill in the art.

Examples of Cations useful within the monoblock include: P Phosphonium, R1 R2M or Morpholinium cation with two alkyl substituents, R1 R2Pip Piperidinium cation with two alkyl substituents, R1 R2Pyr Pyridinium cation with two alkyl substituents, R1 R2Pyrr Pyrolidinium cation with two alkyl substituents, R1 R2R3Im Imidazolium cation with three alkyl substituents, R1 R2R3R4A Ammonium cation with four alkyl substituents, R1 R2R3R4Thi Thiazolium cation with four alkyl substituents, R1 R2R3S Sulphonium cation with three alkyl substituents, R1 R2R3Si Silyl cation with three alkyl substituents, or any other relevant positively charged functional group known by a person of ordinary skill in the art.

### Porous Channel Walls of the Monoblock:

Because the cells have a repeating pattern, the fluid flow channels share common channel walls. These common channel walls, which are made of the same porous material as the monoblock, are also porous and permeable. Depending on the material chosen for the monoblock, the channel walls may also have desirable mechanical, chemical, and thermal properties.

Impregnation with High Boiling Point Liquid:

The porous and permeable channel walls may be impregnated with a high boiling point liquid. Types of high boiling point liquids include organic/fluorinated solvents, Selexol, Rectisol, Purisol, amines, MEA, ammonia, amino acid salts, ionic liquids, enzymes, and other high boiling point liquids that would be known to those skilled in the art.

Impregnating the monoblock with a high boiling point liquid may be accomplished by submersion (dip-coating), vacuum-assist, pressure-assist, or other techniques known in the art to effectuate at least partial pore infusion, and more preferably, complete pore infusion.

The impregnation process may be conducted with various concentrations of the high boiling point liquid depending on the viscosity of the high boiling point liquid and the thickness required for the particular application. Concentration of high boiling point liquid may be 100%, 5-95%, 10-90%, 15-85%, 20-80%, 25-75%, 30-70%, 35-65%, 40-60%, 45-55%, or more preferably, 15-20%.

The concentration of the high boiling point liquid may be adjusted by using a carrier liquid with a lower boiling point than the high boiling point liquid. Such carrier liquids may include an alcohol solution, Diethyl Ether, Acetone, Ethyl Acetate, Hexane, Methyl Ethyl Ketone (MEK), Tetrahydrofuran (THF), Dichloromethane, Chloroform, Carbon Disulfide, Petroleum Ether, or other liquids with lower boiling point than the high boiling point liquid that are known in the art and preferably those that are safe, non-toxic, commercially available, economically feasible, and/or have low greenhouse gas potentials.

Impregnating the monoblock with a diluted form of the high boiling point liquid creates a “thinner” liquid layer, increases permeance into the monoblock material's pores, and reduces the cost for the ionic liquid. In one embodiment, the diluted high boiling point liquid is only applied to the first group of fluid flow channels in the monoblock, which allows for the creation of an asymmetric membrane using the channel wall of the monoblock.

The coating process is typically followed by a process to remove excess high boiling point liquid such that the high boiling point liquid only remains in the pores of the channel walls and does not accumulate in, or block, the fluid flow channels. The subsequent process will also remove the carrier liquid via vaporization, leaving only the high boiling point liquid remaining.

The process to remove excess high boiling point liquid and/or vaporize or evaporate the carrier liquid may include: shaking or spinning the monoblock; directing pressurized air into the fluid flow channels of the monoblock; Ultrasonic cleaning, where the cleaning solution is selected such that it is not a solvent for the high boiling point liquid, but rather merely dislodges the excess high boiling point liquid from the fluid flow channels; manual or automatic brushing or scrubbing; directing pressurized jets of liquid into the fluid flow channels, where the liquid is selected such that it is not a solvent for the high boiling point liquid, but rather merely dislodges the excess high boiling point liquid from the fluid flow channels; applying vacuum or suction to the monoblock; blasting the monoblock fluid flow channels with abrasive materials such as sand or beads propelled by compressed air; directing lasers into the fluid flow channels of the monoblock to selectively evaporate the excess high boiling point liquid that is the fluid flow channels.

### Selection of High Boiling Point Liquids and Ionic Liquids:

The high boiling point liquid may be chosen or designed to have an affinity for a specific fluid species or an affinity for a chemical or physical characteristic shared by multiple fluid species. This affinity between the high boiling point liquid and the target fluid species may include electrostatic forces, ionic affinity, van der Waals forces, hydrogen bonding, covalent bonding, magnetism, opposing polarities.

The chosen high boiling point liquid should have a high boiling point to extend longevity by minimizing loss of the liquid by evaporation over time. The boiling point of the chosen high boiling point liquid should be at least 100° C. The high boiling point liquid may also be selected to have a high affinity to the monoblock material to improve longevity by reducing the loss of high boiling point liquid by mechanical forces such as long-term fluid flow.

The target fluid species may include water vapor, carbon dioxide, deactivated virus, deactivated bacteria, radon, formaldehyde, ethylene, acetaldehyde, acetic acid, acetone, naphthalene, heptane, toluene, carbon monoxide, ammonia, hydrogen sulfide, methanol, SOx, NOx, and/or other hydrocarbons.

One class of high boiling point liquids that are useful for impregnation into the monoblock system are amines. Particular types of amines include monoethanolamine (MEA), methyldiethanolamine (MDEA), 2-Amino-2-methylpropanol (AMP), Piperazine (PIPA), diglycolamine (DGA), diethanolamine (DEA), and di-isopropanolamine (DIPA).

Another class of high boiling point liquids that are useful for impregnation into the monoblock system are ionic liquids. Preferable Ionic liquids have boiling points of at least 100° C., more preferably at least 300° C. to minimize vapor pressure.

Ionic liquids may be selected or formulated to have an affinity for a specific target fluid. Other ionic liquids may be selected or formulated to be a broad-spectrum ionic liquid, meaning it may have been affinity for several types of target fluids, which may or may not share certain chemical or physical characteristics.

Other characteristics of the ionic liquid that may be beneficial for use in the monoblock, depending on the particular application may include ionic conductivity, low vapor pressure, low volatility, high thermal resistance, high electrochemical stability, high solubility of gases and vapors, and/or low flammability.

In an application where CO2 is sought to be separated from the process fluid stream, a preferred ionic liquid would have high CO2 solubility and low absolute enthalpies of solution of CO2, which leads to increased CO2 transported through the monoblock's channel walls and ready desorption of the CO2 on the exhaust fluid stream side.

The low absolute enthalpy of solution of CO2 may correlate with a low viscosity. Amines can be added to ionic liquids and reduce the viscosity of ionic liquids, resulting in faster absorption and mass transfer. In testing, carbon dioxide absorption increased when the amine was mixed with an ionic liquid and impregnated into the monoblock. Carbon dioxide absorption also increased when moisture was present in the amine-ionic liquid solution.

The chosen ionic liquids may be formed by combining a range of anions with large organic cations. One skilled in the art will choose combinations of anions and cations which reduce viscosity, reduce production cost, reduce any potential toxicity, and reduce any potential environmental impact.

If more than one ionic liquid species is impregnated into the monoblock, the individual ionic species' affinities may be retained. In other words, one ionic liquid species may not interfere with the specific or broad-spectrum affinities of another ionic liquid species when two or more ionic liquid species are impregnated in the same monoblock.

When a fluid is absorbed by an ionic liquid, latent heat is released. When a fluid is desorbed from the ionic liquid, latent heat is absorbed. The process is isothermal. If the fluid is in the gas phase when the fluid is absorbed, the fluid will be in the gas phase when the fluid is desorbed from the ionic liquid. A change in partial pressure is one driver behind absorption and desorption. Fluid tends to absorb under higher pressures and desorb under lower pressures.

Some ionic liquids can deactivate microbes and retard growth of biofilm, addressing a problem that has plagued membrane separation techniques.

Ionic liquids that are particularly effective for carbon capture performance may include cations such as ammonium and/or imidazolium and/or anions such as [Tf2N], [BF4], [PF6], [DCA], and/or acetate.

Ionic liquids that are particularly effective for detection of specific gases or vapors such as food aromas, such as beer volatiles, coffee aroma, nitroaromatic explosives, or humidity sensors may include anions such as [Tf2N], [BF4], [PF6], and/or [CI].

Depending on the fluid separation application, the following ionic liquids, combinations thereof, or alterations thereof may be infused into the monoblock: [(SiOSi)C1 MIM][C(CN)3] 1-Methyl-3-pentamethyldisiloxymethylimidazolium Tricyanomethanide, [(SiOSi)C1 MIM][Tf2N] 1-Methyl-3-pentamethyldisiloxymethylimidazolium Bis(trifluoromethylsulfonyl)imide, [APTMS][Ac](3-Aminopropyl)-trimethoxysilane Acetate, [BMIM][Ac] 1-Butyl-3-methylimidazolium Acetate, [BMIM][BF4] 1-Butyl-3-methylimidazolium Tetrafluoroborate, [BMIM][DCA] 1-butyl-3-methylimidazolium Dicyanamide, [BMIM][Doc] 1-Butyl-3-methylimidazolium bis(2-ethylhexyl)sulfosuccinate, [BMIM][Pho] 1-Butyl-3-methylimidazolium Phenolate, [BMIM][Tf2N] 1-butyl-3-methylimidazolium Bis(trifluoromethylsulfonyl)imide, [BMIM][TfO]1-butyl-3-methylimidazolium Trifluoromethane sulfonate, [C1C3MIM][Tf2N] 1-methyl-3-propylmethylimidazolium Bis(trifluoromethylsulfonyl)imide, [C3C1MIM][Tf2N] 1-propyl-3-methylimidazolium Bis(trifluoromethylsulfonyl)imide, [Choline][Gly] Cholinium Glycolate, [Choline][Lac] Cholinium L(+) lactate, [Choline][Lev] Cholinium Levulinate, [Choline][Mal] Cholinium Malonate, [Choline][Pro] Cholinium Proline, [DMAPAH][EtOAc]3-(Dimethylamino)-1-propylammonium Ethoxyacetate, [DMAPAH][TfA] 3-Dimethylamino-1-propylammonium Trifluoroacetate, [EMIM][Ac] 1-Ethyl-3-methylimidazolium Acetate, [EMIM][C(CN)3] 1-Ethyl-3-methylimidazolium Tricyanomethanide, [EMIM][ESU] 1-Ethyl-3-methylimidazolium Ethyl sulfate, [EMIM][Et2PO4] 1-Ethyl-3-methylimidazolium Diethylphosphate, [EMIM][FAP] 1-Ethyl-3-methylimidazolium Trifluoro phosphate, [EMIM][Me2PO4] 1-Ethyl-3-methylimidazolium Trimethylphosphate, [EMIM][Tf2N] 1-Ethyl-3-methylimidazolium Bis(trifluoromethylsulfonyl)imide, [EMIM][TfA] 1-Ethyl-3-methylimidazolium Trifluoroacetate, [HMIM][Tf2N] 1-Hexyl-3-methylimidazolium Bis(trifluoromethylsulfonyl)imide, [HmMIM][Tf2N] 1-Hexyl-2-methyl-3-methylimidazolium Bis(trifluoromethylsulfonyl)imide, [OMIM][Ac] 1-Octyl-3-methylimidazolium Acetate, [P222(101)][Inda] Triethyl(2-methoxymethyl) phosphonium Indazolide, [P2225][Pro] Triethyl(pentyl)phosphonium Prolinate, [P4444][Pro] Tetrabutylphosphonium Prolinate, [Si-C1-C3-MIM][Tf2N] 1-Methyl-3-(2-methyl-3-(trimethylsilyl)propyl)imidazolium Bis(trifluoromethylsulfonyl)imide, [VBTMA[Ac] Vinylbenzyl trimethylammonium Acetate, [VOIM][PF6] 1-vinyl-3-octylimidazolium Hexafluorophosphate, 6FDA-TeMPD (Hexafluoroisopropylidene)dephthalic anhydride and 2,3,5,6-tetramethyl-1,4-phenylene diamine-based polyimide, p(VDF-HFP) Poly(vinylidene fluoride-co-hexafluoropropylene) fluoroelastomer, p[MABI][BF4] Poly(1-[2-(methacryloyloxy)ethyl]-3-butyl-imidazolium) tetrafluoroborate, p[MATMA][BF4] poly[2-(methacryloyloxy)ethyl]trimethylammonium tetrafluoroborate, p[VBBI][BF4] Poly(1-(p-vinylbenzyl)-3-butyl-imidazolium) tetrafluoroborate, p[VBIM][DCA] 1-vinyl-3-butylimidazolium dicyanamide, p[VBTMA][BF4] poly[1-(para-vinylbenzyl)-triethylammonium tetrafluoroborate, p[VBTMA][PF6] poly[1-(para-vinylbenzyl)-triethylammonium hexafluorophosphate, p[VBTMA][Tf2N] poly[1-(para-vinylbenzyl)-triethylammonium bis(trifluoromethylsulfonyl)imide, p[VEIM][DCA] polymerized 1-vinyl-3-ethylimidazolium dicyanamide, p[VHIM][DCA] 1-vinyl-3-heptylimidazolium dicyanamide, P4VP Poly(4-vinylpyridine), PAA Poly acrylic acid or Sodium polyacrylate, PAH Poly(allylamine) hydrochloride, PAMPS Poly(2-acrylamido-2-methyl-1-propanesulfonic acid), PAN Polyacrylonitrile, PBI Polybenzimidazole, PC Polycarbonate, PDADMAC Poly(diallyldimethylammonium) chloride, PDMAAm Polydimethylacrylamide, PDMAEMA Poly(N,N-dimethyl aminoethylmethacrylate), PDMS Polydimethylsiloxane, PEG Polyethylene glycol, PEI Poly(ethyleneimide), PES Polyethersulfone, PI Polyimide, PMAPTAC Poly(methacryloylamino propyl trimethylammonium chloride), PMDA-ODA PI Poly(pyromellitimide-co-4,40-oxydianiline) polyimide, PMMA Polymethylmethacrylate, Poly(SEOS) Poly(styrene-block-ethylene oxide-block-styrene), PSf Polysulfone, PSS Polystyrene sulphonate or Sodium polystyrene sulphonate, PTFE Polytetrafluoroethylene, PTMEG Polytetramethylene Ether Glycol, PTMSP Poly(1-trimethylsilyl-1-propyne), PU Polyurethane, PVAc Poly(vinyl) acetate, PVBC Polyvinyl benzyl chloride, PVBTMAC Poly(4-vinylbenzyltrimethylammonium) chloride, PVDF Polyvinylidene fluoride, and PVP Polyvinylpyrrolidone.

Various types of ionic liquids and the fluid species with which they have a particular affinity are shown in Table 1 below.

TABLE 2

| Ionic Liquid | Fluid Species |
|---|---|
| [EMIM][Tf2N] | CO2 CH4, CO2 |
| [EMIM][TfA] | CO2 CH4, CO2 |
| [BMIM][Tf2N] | CO2 H2, N2, CH4, CO, CO2 |
| [C3C1MIM][Tf2N] | CO2 |
| [BMIM][Ac] | CO2 |
| [Vbtma][Ac] | CO2 |
| [EMIM][Ac] | CO2 |
| [Si-C1-C3-MIM][Tf2N] | CO2 |
| [BMIM][Pho] | CO2 |
| [EMIM][BF4] | CO2 & H20 H2, N2, CH4, |
| [Choline][Pro]/PEG200 1:0 | CO2 |
| [Choline][Lev] | CO2 |
| [Choline][Lac] | CO2 |
| [Choline][Gly] | CO2 |
| [Choline][Mal] | CO2 |
| [BMIM][BF4] | He, H2, O2, N2, CH4, CO2, |
| | CO2/N2, CO2/CH4 |
| [BMIM][doc] | CO2 |
| [DMAPAH][EtOAc] | CO2 |
| [DMAPAH][TFA] | CO2 |
| [(SiOSi)C1MIM][Tf2N] | CO2 |
| [(SiOSi)C1MIM][C(CN)3] | CO2 |
| [BMIM][DCA] | CO2 H2, N2, O2, CO2, CH4 |
| [BMIM][Ac] | CO2 & H2S & CH4 |
| [BMIM][TfO] | CO2 & CH4 |
| [BMIM][Tf2N] | He, H2, O2, N2, CH4, |
| | CO2/N2, CO2/CH4 |
| [EMIM][TfO] | CO2 & CH4 |
| [Ch][Lev] | CO2 & CH4 |
| [Ch][Lac] | CO2 & CH4 |
| [Ch][Gly] | CO2 & CH4 |
| [Ch][Mal] | CO2 & CH4 |
| [BMIM][BF4] | CO2 & H2 & H2S & CH4 |
| [OMIM][Ac] | CO2 & CH4 |
| [APTMS][Ac] | CO2 & CH4 |
| [DMAPAH][EtOAc] | CO2 & CH4 |
| [DMAPAH][TFA] | CO2 & CH4 |
| [EMIM][FAP] | CO2 & CH4 |
| [EMIM][DCA] | H20 & VOCs H2, N2, O2, |
| | CO2, CH4 |
| [EMIM][ESU] | H20 & VOCs |
| CuCl/[BMIM][Cl] | Ethylene/ethane & VOCs |
| [EMIM][Me2PO4] + Ag | Ethylene/ethane & VOCs |
| [EMIM][Et2PO4] + Ag | Ethylene/ethane & VOCs |
| [HMMIM][Tf2N] + Ag carriers | Propylene/propane & VOCs |
| [HMIM][Tf2N] + Ag carriers | Propylene/propane & VOCs |
| [BMPyr][DCA] | H2, O2, CO2, CH4 |
| [EMIM][BF4] | O2, CH4, CO2 |
| [C2MIM][Tf2N] | O2, CH4, CO2 |
| [C4MIM][Tf2N] | O2, CH4, CO2 |
| [C6MIM][Tf2N] | O2, CH4, CO2 |
| [BMIM]2[Co(NCS)4] (on PAN) | O2 |
| [Methylammonium] | O2, CO2 |
| [Diisopropylamonium] | O2, CO2 |
| [N,N-diisopropylethylamonium] | O2, CO2 |
| [EMIM][B(CN)4] | H2, CO2 |
| [BMIM][PF6] | H2, CO2 |
| [BMIM][Tf2N] | H2, CO2 |
| [BMIM][B(CN)4] | H2, CO2 |
| [EMIM][TFSI] | He, H2, O2, N2, CH4, CO2, |
| | Butane, pentane, hexane, |
| | cyclohexane, heptane, |
| | toluene, MeOH, EtOH, |
| | PrOH, BuOH, H2O |
| [HMIM][Tf2N] | CH4, CO2, EtOH, Hexane, |
| | isooctane |
| [EMIM][TFSI] | He, H2, O2, N2, CH4, CO2 |
| | R-(+)- limonene/S-(+)- |
| | carvone/EtOH(/H2O) |
| [HdMIM][TFSI] | He, H2, O2, N2, CH4, CO2 |
| | R-(+)- limonene/S-(+)- |
| | carvone/EtOH(/H2O) |
| [BMIM][CF3SO3] | He, H2, O2, N2, CH4, CO2 |
| [TETA][Tfa] | CO2, N2 |
| [EMIM][EtSO4] | H2S, CH4, CO2, Air |
| [EMIM][Gly], [BMIM][Gly], | N2, CH4, CO2/N2, CO2/CH4 |
| [HMIM][Gly], [BMIM][Ac], | |
| [BMIM][BF4] | |
| [EMIM][SCN], [EMIM][BF4], | CH2CF2 (R32), CF3CHF2 (R125) |
| [EMIM][OTf], [EMIM][Tf2N] | CH2CF2/CF3CHF2 (R410A) |
| [P4444][Gly], [P4444][Ser], | N2, CO2 |
| [P4444][Lys], [P4444][Pro] | |

Phosphonium-based ionic liquids have certain characteristics that make them preferable for certain applications. Those characteristics include minimal release of VOCs into the conditioned process fluid stream and the exhaust fluid stream; ability to absorb CO2 and all fourteen ASHRAE Standard 62.1-2022 pollutant design compounds for indoor air quality even after being directly exposed to liquid water; and low Henry's Law constants, making them good absorbents. Further, Phosphonium-based ionic liquids have a highly stable structure, allowing them to absorb pollutants from high-temperature applications. Due to this stability, they have been shown service lives of over ten years in extreme applications such as dryer and press exhaust gas applications.

Tetrabutylephosphonium levulinate is an ionic liquid with a high CO2 absorption rate at low pressure, with a capacity of 1.5 mmol/g at 303 K and 2 bar, similar to those of sorbent materials such as for N-ethyl-diethanolamine (DEA) (≈1.8 mmol/g at 40° C. and 1 bar) or solid amine sorbents such as mesoporous silica along with polyethylenimine (2.6 mmol/g at 30° C. and 1 bar).

Tetrabutylephosphonium levulinate functions via physical surface area (non-chemical) bonding and thus also exhibits one of the lowest regeneration energies compared to other high boiling point liquids. Further, Tetrabutylphosphonium levulinate's viscosity of 253 cP at room temperature is beneficial for the energy transfer characteristics of the monoblock.

Furthermore, the monoblock was tested using tetra-n-butylphosphonium-based amino acid ionic liquids including with L-glycinate, L-alaninate, L-serinate, and L-prolinate as anions. Some of these exhibited CO2 permeability and C02/N2 selectivity above the Robeson upper bounds.

Ionic liquid EMIM BF4 has a high water permeance value at 1.6 E\hat{ }-6.

Mixing multiple ionic liquids is another method of fine-tuning absorption properties. An exemplary mixture of ionic liquids would comprise N-(2-aminoethyl)ethanolamine-based IL ([AEEA][X]) and 1-ethyl-3-methylimidazoliumacetate([emim][AcO]). This mixture is a liquid at temperatures below 0° F., have negligible vapor pressure and thus negligible loss out of the monoblock system, and are inert to most chemicals found in a process fluid stream. The two ionic liquids exhibited service lives of over 15 years at prolonged temperatures over 400° C. the mixture shows high CO2 absorption rates and low absolute enthalpy of CO2 solution. Without seeking to be limited by theory, the mechanism of the mixture to absorb CO2 is theorized to be as follows: [AEEA]-[X] acts as a CO2 carrier, whereas the Lewis basic [emim][AcO] stabilizes zwitterion/carbamic acid species that exhibit relatively low absolute enthalpy of solution as compared to carbamate species. Testing results show a CO2 permeability greater than 26,000 Barrer and a C02/N2 selectivity of greater than 10,000.

Ionic liquid, 1-butyl-3-methyl-imidazoliumdicyanamide ([bmim][DCA]) has the ability to capture both helium and carbon dioxide from a feed stream.

Ionic liquids may also be useful for separating radiochemical compounds including lanthanides, actinides, and fission products. Ionic liquids chosen for this application would preferably have low vapor pressures, high combustion resistance, and wide electrochemical windows. RTILs with altered side-chain functional groups have shown promise as “task specific” ligands for selective partitioning of metal ions. For example, imidazolium cation functionalized with the uranium-selective amidoxime functional group infused into the monoblock may advantageously capture radioactive material in a process fluid stream. Ionic liquids based on boron-containing anions have also shown promise for this application because they confer intrinsic safety against criticality accidents. The hydrophobic ionic liquid 1-butyl-3-methylimidazolium bis(trifluoromethylsulfonyl)-imide [C4mim][Tf2N] may be used as a diluent for β-diketone lanthanide extraction. Commonly available imidazolium anions include [Tf2N]−, [PF6]−, and [BF4]−.

Ionic liquids suitable for oxygen separation may include: Co(II) chelated ionic liquids such as [P66614]2[NmGly]2[Co(salen)], [P66614]2[NmGly][Tf2N][Co(salen)], [Bmim][PF6], and/or other ionic liquids depending on the application.

Ionic liquids suitable for NOx and SOx separation may include: [P66614][Tetz], imidazolium-based ionic liquids, ionic liquids functionalized with anions (e.g., [FeCl4]2−), amine groups, sulfonate and carboxylate anions, or a combination of superbase ionic liquids with a trihexyltetradecylphosphonium cation and a benzimidazolide ([P66614][Benzim]) or tetrazolide ([P66614][Tetz]) anion, and/or other ionic liquids depending on the application.

### Fluid Separation by the Monoblock:

The number of open cells on the front face of the monoblock, N, may be divided into two or more groups. For example, there may be first and second groups of fluid flow channels. In some embodiments, the number of fluid flow channels for the first and second groups of fluid flow channels may each be about N/2. At least a portion of the first group of fluid flow channels is adjacent to at least a portion of the second group of fluid flow channels. For example, each of the fluid flow channels making up the first group of fluid flow channels may be adjacent to at least one of the fluid flow channels making up the second group of fluid flow channels.

A process fluid stream comprising several species of fluids, including one or more target fluids, enters the first group of fluid flow channels on the front face of the monoblock, optionally, under the influence of positive pressure created by a pump. Any fluid pump known in the art may be used. Or the process fluid stream may enter the first group of fluid flow channels after being generated by a chemical process, for example, a flue-gas stack. In one embodiment, the volumetric flow rate of the process fluid stream is about 9 cfm.

As the process fluid stream flows through the first group of fluid flow channels, one or more target fluid species within the process fluid stream become attracted to the high boiling point liquid impregnated in the channel. The high boiling point liquid will then absorb at least a portion of the target fluid species.

At least one fluid flow channel from the second group of fluid flow channels is adjacent to, and thus shares a common channel wall with, at least one fluid flow channel from the first group of fluid flow channels.

The monoblock system uses chemical gradient, namely the concentration or pressure gradient between two surfaces of the monoblock wall, to separate fluid species from the process fluid stream across the channel wall to the exhaust side. Four distinct techniques may be used to create the partial vapor pressure difference: feed compression, vacuum pumping, gas sweep, and a combination of vacuum pumping and gas sweep.

Feed compression uses a compressor to increase the pressure in the process fluid stream or first group of fluid flow channels. Vacuum pumping uses a vacuum pump to reduce the pressure in the exhaust fluid stream or second group of fluid flow channels. Both feed compression and vacuum pumping requires energy to produce either higher or lower pressure.

Alternatively, gas sweep adds an inert gas to the exhaust fluid stream or second group of fluid flow channels for the diluting, resulting in a lower partial vapor pressure. Gas sweep operates the second group of fluid flow channels at a higher total pressure and requires an additional compound like inert gas, nitrogen, or air, which can be costly. Various embodiments of the monoblock separation systems may employ a combination of vacuum pump and gas sweep or a combination of compression feed and vacuum pump.

The pressure differential on either side of the channel walls is a driver behind absorption and desorption of the target fluid species. Therefore, the target fluid species will be absorbed on the higher pressure side of the channel wall, and desorbed on the lower pressure side.

In other words, once the target fluid species is absorbed by the high boiling point liquid, the target fluid species will diffuse through the high boiling point liquid from a side with a higher target fluid species partial pressure, to a side with a lower target fluid species partial pressure. Therefore, the absorbed target fluid species will diffuse from the process fluid stream side of the channel wall to the exhaust fluid stream side of the channel wall due to the associated high-to-low partial pressure gradient.

A vacuum may compress air from 1 kPa to a standard atmospheric pressure of 101 KPa. The work, W, required by the vacuum is calculated using the following formula: W=(nRT/E)·ln (Pₒᵤₜₗₑₜ/Pᵢₙₗₑₜ). In this equation, n is the total moles of the mixture vacuumed by the pump, R is the gas constant, T is the absolute temperature, typically Pₒᵤₜₗₑₜ is the atmospheric pressure, Pᵢₙₗₑₜ is the exhaust fluid stream pressure, and E is the efficiency of the vacuum pump, which can be assumed as 0.65. The compression ratio of the vacuum pump, (Pₒᵤₜₗₑₜ/Pᵢₙₗₑₜ), determines the magnitude of the work.

To reduce energy requirement from the vacuum pump, an embodiment includes a vacuum pump located between two monoblocks operating in series. The low-pressure side of the vacuum pump was designed for the partial pressure of the exhaust fluid stream to be less than 1.25 kPa from the exhaust fluid stream side of the first monoblock and the high-pressure side of the vacuum pump is at a partial pressure of the exhaust fluid stream of 3.5 kPa, which is the feed side of the second monoblock. In this configuration, the vacuum pump does not need to compress the exhaust fluid stream side of the first monoblock up to the ambient pressure, but at a much lower partial pressure of the exhaust fluid stream of 3.5 kPa. Thus, in this embodiment, the work used for the vacuum pump is only about one-third of the work used for a typical compression ratio at 100 because of the much smaller compression ratio of three to five.

Fluid exits the monoblock in two forms: as a conditioned process fluid stream and as an exhaust fluid stream.

The remaining portion of the process fluid stream that does not get absorbed by the high boiling point liquid (“the conditioned process fluid stream”) continues to flow through the first group of fluid flow channels, which are open on the rear face of the monoblock. The conditioned process fluid stream thus exits through the rear face of the monoblock. In one embodiment, the process fluid stream flowing through the monoblock experiences a relatively low-pressure drop due to laminar flow through the monoblock. Therefore, the monoblock conditions the process fluid stream by separating the target fluid species from the process fluid stream.

After the target fluid species gets desorbed from the high boiling point liquid on the lower pressure side of the channel wall, the separated target fluid species (“the exhaust fluid stream”) flows into the second group of fluid flow channels. The exhaust fluid stream then flows through the second group of fluid flow channels and exits the monoblock.

In one embodiment, one end of the second group of fluid flow channels is restricted. This restricted end of the second group of fluid flow channels allows a vacuum pump optionally connected to the unrestricted end of the second group of fluid flow channels to generate negative pressure more effectively in those channels to extract the exhaust fluid stream from the monoblock. The restricted end may take the form of a fluid-tight seal.

In one embodiment, the front face of the second group of fluid flow channels is restricted and the exhaust fluid stream exits the monoblock on the rear face of the monoblock. In this embodiment, a header, as more thoroughly described below, is optionally connected to the rear face of the monoblock to separate the exhaust fluid stream from the conditioned fluid stream. In this embodiment, the end of the second group of fluid flow channels on the front face of the monoblock may be restricted. This restricted end of the second group of fluid flow channels allows a vacuum pump optionally connected to the rear face of the second group of fluid flow channels to generate negative pressure more effectively.

In another embodiment, the rear face of the second group of fluid flow channels is restricted and the exhaust fluid stream exits the monoblock on the front face of the monoblock. In this embodiment, a header, as more thoroughly described below, is optionally disposed on the front face of the monoblock to facilitate input of the process fluid stream into the first group of fluid flow channels and facilitate removal of the exhaust fluid stream from the second group of fluid flow channels. In this embodiment, the exhaust fluid stream and process fluid stream flow countercurrent to one another.

A countercurrent flow may be more efficient than a co-current exchange because a countercurrent flow maximizes exchange duration while providing a consistent gain/loss gradient during the exchange process. For example, at the portion of the monoblock closest to the front face, the process fluid stream has its highest concentration of the target fluid species, and the exhaust fluid stream is also at its highest concentration of the target fluid species. At the portion of the monoblock closest to the rear face, the process fluid stream has its lowest concentration of the target fluid species, and the exhaust fluid stream also has its lowest concentration of the target fluid species. This counterflow exchange therefore maximizes the difference of partial pressure of the target fluid species across the channel walls throughout the length of the monoblock. In this embodiment, the ends of the second group of fluid flow channels on the rear face of the monoblock may be restricted to allow a vacuum pump optionally connected to the second group of fluid flow channels to generate negative pressure more effectively.

In yet another embodiment, the middle of the second group of fluid flow channels is restricted. In this embodiment, the exhaust fluid stream and process fluid stream flow both countercurrent and co-current to one another. In this embodiment, in the first half of the monoblock, before the restriction in the second group of fluid flow channels, a process fluid stream is introduced to the first group of fluid flow channels and a subset of the target fluid species within the process fluid stream is transferred through the channel walls into the second group of fluid flow channels and is extracted through the front face of the monoblock, using, optionally, vacuum pressure. After an initial separation pass, the conditioned process fluid stream passes the halfway point of the monoblock, which is where the second group of fluid flow channels is restricted. A subset of the target fluid species that was not separated from the process fluid stream in the first half of the monoblock's transfer through the channel walls into the second half of the second group of fluid flow channels and is extracted through the rear faces the monoblock, using, optionally, vacuum pressure. Therefore, in the first half of the monoblock, the exhaust fluid stream flows counter-current to the process fluid stream, and in the second half of the monoblock, the exhaust fluid stream flows co-current to the process fluid stream. A header disposed on the front face of the monoblock delivers the process fluid stream and extracts the first exhaust fluid stream. A second header disposed on the rear face of the monoblock extracts both the conditioned process fluid stream and the second exhaust fluid stream.

### Monoblock Housing:

In an application where the continuous conditioning of a process fluid stream is desirable, a fluid conditioning unit (“FCU”) may be provided, comprising an array of monoblocks to condition a process fluid stream in parallel. An FCU may also comprise pipes or tubing needed to route fluid flow, sensors, filters disposed upstream and/or downstream of the monoblock, a power source, scaffolding to organize and support the monoblock array, distribution fans, pumps, and other components that would be apparent to a person of skill in the art.

A housing may be provided to enclose and seal at least one monoblock, a header, and any piping and channeling necessary for delivering and removing fluid streams to and from the monoblock. In this arrangement, a single housing is a line replaceable unit (“LRU”) and can be replaced individually during the process fluid stream conditioning operation in the event of component failure or for routine maintenance. The housing may be openable for access to the enclosed monoblock and header.

The housing may provide one or more ports for: the process fluid stream, the conditioned process fluid stream, and/or the exhaust fluid stream. Standard fluid flow ports that are known in the art may be used.

The housing may have connecting mechanisms such as snaps, clips, latches, or any other mechanism or substance to removably connect to adjacent housing(s) and/or a structural scaffolding.

Each LRU may incorporate sensors to monitor the performance and operation of the monoblock incorporated within that housing. The sensors may communicate with a central processing unit (“CPU”) to send an alert when an LRU needs replacement, which may be performed automatically or manually. The CPU also may connect to a central controller in order to divert the process fluid stream to other LRUs when one needs replacement. This diverting process may be conducted by controlling fans or pumps connected to each LRU or by actuating dampeners or baffles to physically block fluid flow from entering the LRU in need of replacement.

### Header:

As previously explained, the process fluid stream enters the front face of the monoblock and exits the rear face of the monoblock. The exhaust fluid stream may exit the monoblock through the front face or the rear face of the monoblock, which results in either counter-current or co-current flow, respectively.

In the absence of a header, a plenum, wherein a structure directs a flow of fluid towards one side of the monoblock without routing that fluid into any particular fluid flow channels in the monoblock, may be used.

If the cells take the shape of a rectangle, the inlet and outlet fluid flow channels may be arranged in a checkerboard pattern, such that each inlet fluid flow channel is surrounded by outlet fluid flow channels, and vice-versa. In a complex arrangement such as the checkerboard arrangement, a header may be used to route the process fluid stream into the first group of fluid flow channels and route the exhaust fluid streams out of the second group of fluid flow channels, such that the process fluid and exhaust fluid streams do not mix.

A header may be provided to transition a fluid stream between bulk flow outside the monoblock and multi-channel flow within a first or second group of fluid flow channels of the monoblock. Such a header may perform two distinct jobs: first, transition a fluid stream at side A of the header between a bulk flow and a multi-channel flow, and second, routing the multi-channel flow of the fluid stream into or out of the relevant fluid flow channels of the monoblock.

The partitioner portion of the header performs the first job. The partitioner may have any port known in the art to intake or output a fluid stream at Side A of the header. The partitioner transitions the fluid stream between bulk flow and a multi-channel flow at a transition point between the partitioner and the channelizer portions of the header. The number of channels at the transition point would approximately equal the number of fluid flow channels in the monoblock. In one embodiment, the multi-channel flow created by the partitioner is in a laminar flow regime.

The channelizer portion performs the second job. The channelizer portion of the header either 1—routes the multi-channel flow of process fluid into the first group of fluid flow channels in the monoblock or 2—extracts the multi-channel flow of exhaust fluid out of the second group of fluid flow channels in the monoblock to the partitioner portion of the header. The number and arrangement of cells on the side of the channelizer opposite the partitioner (“Side B of the header”) are approximately the same as the cells on the monoblock.

If a header is used to remove the exhaust fluid stream from the front face of the second group of fluid flow channels of the monoblock, a header or a plenum may be used to route the process fluid stream into the first group of fluid flow channels. Alternatively, if a header is used to input the process fluid stream into the first group of fluid flow channels of the monoblock, a header or a plenum may be used to remove the exhaust fluid stream from the front face of the second group of fluid flow channels.

Side B of the header may be attached to either or both of the faces of the monoblock. The header may be removably attached to the monoblock via a connection mechanism, such as snaps, clips, screws, latches, a removable adhesive, or any other mechanism or substance to removably connect the header to the monoblock. Or the header may be permanently attached to the monoblock via, for example, a permanent adhesive, rivets, by being formed directly onto the monoblock structure, or any other mechanism or substance to permanently connect the header to the monoblock.

The geometry and material of the header structure should preferably minimize the pressure drop between the bulk flow of fluid and the resulting multi-channel flow of fluid.

The header may be manufactured by micro laser drilling, micro CNC milling, 3D printing, micro injection molding, or urethane casting, or any other method known to a person of ordinary skill in the art.

Regarding manufacturing of the header using micro laser drilling or micro CNC milling: All classes of materials can be laser drilled including ceramics, polymers, semiconductors, glasses, and metallic materials. Femto-second and Nano-second lasers use a high power density to vaporize square channels including single pulse drilling, percussion drilling, trepanning, and helical drilling. Advantages ensuing from the use of laser drilling include non-contact with header, higher accuracies, and higher machining rates. Non-limiting examples of such materials include: EP (epoxy resin), ABS, PBT, PA, Polycarbonate, Polypropylene, Polyethylene, PPS, Polystyrene, Polyimide, Polyvinyl chloride, Glass epoxy, Stainless Steel, Iron, Aluminum, Nickel, Copper, Brass, Bronze, Ceramic, Carbon, Activated carbon, Activated alumina, PEEK, Polyetherimide (PEI), Polyether Ether Ketone (PEEK), Polybenzimidazole (PBI), Polydicyclopentadiene (pDCPD).

Regarding manufacturing of a header using 3D printing: SLA 3D printing may be conducted using various blends of monomers, oligomers, photo initiators, and other additives that result in different material properties based on the intended application of the monoblock. Thermoset polymers include PA, PLA, and ABS. Porous ceramics include activated carbon and activated alumina. Additional 3D print materials include aluminum, stainless steel, titanium, HIPS, PETG, nylon, carbon fiber, ASA, polycarbonate, polypropylene, metal-filled, wood-filled, carbon-filled, PVA, and other materials that have been or can be used in 3D printing applications.

Manufacturing a header using micro injection molding may use materials including: polyethylene, polypropylene, nylon, polycarbonate, Delrin, polysulfone, polybutylene terephthalate, acrylic, peek, Ultem, liquid crystal polymer.

Manufacturing a header using urethane casting may use materials including polymers, biodegradable polymers, metals, and other materials that may be poured into a negative mold and set.

### Multi-Monoblock Operation for Conditioning Building Air:

When multiple monoblocks are utilized in a single system, multiple aspects of building air can be conditioned simultaneously.

In one embodiment where decreased water vapor concentration is desired inside the building, a first monoblock can be impregnated with a liquid with a high affinity for water vapor and a second monoblock can be impregnated with a liquid with a high affinity for CO2. When used in series, the first monoblock can intake indoor air and the separated water vapor can be exhausted to the outdoors. The second monoblock can then intake the conditioned process fluid stream from the first monoblock and reduce the CO2 concentration in the air. The separated CO2 can then be exhausted to the outdoors, used downstream, or sequestered. The resulting conditioned process air stream with reduced water vapor and CO2 concentration can then be returned to the building.

In another embodiment where increased water vapor concentration is desired inside the building, the first monoblock impregnated with a liquid with a high affinity for water vapor intakes outdoor air, separates a portion of the water vapor in the outdoor air, and exhausts that water vapor into the building, thereby increasing the water vapor concentration in the building air.

In another embodiment, the second monoblock can be impregnated with a liquid with a high affinity for CO2 and other indoor air pollutants.

In another embodiment, a third monoblock can be impregnated with a liquid with a high affinity for indoor air pollutants with relevant regulatory standards that permit the partial or complete elimination of outdoor air in building ventilation systems. Those indoor air pollutants and their associated maximums, according to ANSI/ASHRAE Standard 62.1-2022 are reproduced in Table 2 below.

TABLE 3
Design Compounds, PM2.5, and Their Design limits

| | | |
|---|---|---|
| Compound or PM2.5 | Design limit | Removal Efficiency |

ANSI/ASHRAE Standard 62.1-2022

| | | | |
|---|---|---|---|
| Acetaldehyde | 140 | μg/m\hat{ }3 | 99% |
| Acetone | 1,200 | μg/m\hat{ }3 | 99% |
| Benzene | 3 | μg/m\hat{ }3 | 87% |
| Dichloromethane | 400 | μg/m\hat{ }3 | 54% |
| Formaldehyde | 33 | μg/m\hat{ }3 | 99% |
| Naphthalene | 9 | μg/m\hat{ }3 | 87% |
| Phenol | 10 | μg/m\hat{ }3 | 60% |
| Tetrachloroethylene | 35 | μg/m\hat{ }3 | 54% |
| Toluene | 300 | μg/m\hat{ }3 | 52% |
| 1,1,1-trichloroethane | 1000 | μg/m\hat{ }3 | 54% |
| Xylene, total | 500 | μg/m\hat{ }3 | 60% |
| Carbon monoxide | 9 | ppm | 99% |
| PM2.5 | 12 | μg/m\hat{ }3 | MERV 11 |
| Ozone | 70 | ppb | 70% |
| Ammonia | 200 | μg/m\hat{ }3 | 99% |

Not Required by ASHRAE Standard 62.1-2022

| | | |
|---|---|---|
| Carbon Dioxide | — | 80% |
| Nitrogen Dioxide | — | 94% |
| SARS-CoV-2 | — | 76% |

(Efficiencies based on ASHRAE 145.2 and 52.2 test methods)
(Published Amine (MEA) and Ionic liquid Removal Rates)

In another embodiment, a monoblock can be paired with heated or cooled fluid to heat or cool the building air. Heating or cooling of the fluid stream may be accomplished by tubes carrying heated or cooled fluid being inserted into one or more of the fluid flow channels such that the tubes contact the channel walls, but do not fill the volume of the fluid flow channels. Heat is then able to be exchanged between the fluid in the tubes and the building air flowing through the monoblock.

Alternatively, the channel walls of one or more fluid flow channels of the second group of fluid flow channels may be glazed, and heated or cooled fluid may be pumped through the fluid flow channels with glazed channel walls such that heat flows through the monoblock's thermally conductive channel walls to condition the temperature of the building air. Alternatively, the monoblock may be placed on a heating or cooling surface, which will heat or cool the channel walls, resulting in heat transfer between the fluid streams and the channel walls.

Alternatively, the monoblock may be an electrocaloric device made of a ferromagnetic material. The electrocaloric monoblock is then exposed to an electric field, which triggers changes in the material's polarization, so that applying an electric field increases the material's temperature, whereas its removal induces cooling. See Jaka Tusek, A Highly Efficient Solid-State Heat Pump, 383 Sci. 769, 769-770 (Nov. 17, 2023). Once the monoblock material heats or cools, heat is transferred between the fluid streams and the channel walls.

Therefore, one monoblock may both adjust the temperature of and the concentration of target fluids in a process fluid stream.

Consistent with the foregoing applications, in one embodiment, a header configured to divide a bulk flow of fluid into a multi-channel flow of fluid is disclosed. A bulk fluid may include air from the atmosphere, or an enclosed environment, such as a building, an air-tank, or a vehicle, including automobile, submersible, or airplane. The header configured to divide a bulk flow of fluid into a multi-channel flow of fluid may include at least one inlet, a transition element, a plurality of routing channels, and at least one outlet. In some embodiments, the at least one outlet comprises a plurality of openings that are fluidly connected to a plurality of fluid flow channels on a device attached to the header. In some embodiments, the transition element is configured to divide the first bulk flow of fluid into a first multi-channel flow of fluid. In some embodiments, a plurality of first routing channels is configured to route the first multi-channel flow of fluid to a first outlet. In some embodiments, a first outlet comprises a plurality of openings that are fluidly connected to a first group of fluid flow channels.

In some embodiments, the header described herein may further comprise: a second inlet comprising a plurality of openings that are fluidly connected to a second group of fluid flow channels on the device attached to the header. The second inlet is typically configured to receive a second multi-channel flow of fluid from the second group of fluid flow channels. In this embodiment, the second inlet is configured to receive a second multi-channel flow of fluid from the second group of fluid flow channels. In some embodiments, a plurality of second routing channels are configured to route the second multi-channel flow of fluid from the second inlet to the transition element. In some embodiments, the transition element is configured to merge the second multi-channel flow of fluid into a second bulk flow of fluid. In some embodiments, a second outlet is configured to exhaust the second bulk flow of fluid from said header. In some embodiments, the first group of fluid flow channels is structurally distinct from the second group of fluid flow channels, and the first routing channels are structurally distinct from and not in fluid communication with the second routing channels.

In some embodiments, the header described herein may be configured to maintain a pressure differential between the first group of fluid flow channels and a second group of fluid flow channels.

In some embodiments, the header may further comprise: a plenum structure and a second inlet that is fluidly connected to a second group of fluid flow channels on the device attached to the header, wherein the second inlet is configured to receive a second multi-channel flow of fluid from the second group of fluid flow channels. In some embodiments, the plenum structure is configured to merge the second multi-channel flow of fluid to form a second bulk flow of fluid, a second outlet is configured to exhaust the BF2, and the first group of fluid flow channels is structurally distinct from the second group of fluid flow channels. As used herein, a plenum structure generally refers to a space that serves as a pathway for the distribution of air. In HVAC systems, for example, a plenum is an enclosed space used for air distribution or for housing HVAC components. The plenum helps ensure the even distribution of conditioned air throughout the building. In some embodiments, the plenum structure is selected from a group consisting of a housing, a manifold, ductwork, a chamber, an air distribution box, and a cavity.

In some embodiments, the header is at least partially made of a polymer, dissolvable polymer, wax, metal, or a combination thereof. Non-limiting examples of the polymeric material that may be used herein include polyethylene, polypropylene, and polyvinyl chloride (PVC). Non-limiting types of composites that may be used herein include fiber-reinforced composites, such as a polymer matrix reinforced with fibers (e.g., carbon fiber, glass fiber), or metal matrix composites, such as a combination of metals and ceramic materials.

In some embodiments, the first and second groups of fluid flow channels (FC1 and FC2) on the device comprises: channels between 200 and 1200 channels per square inch, and lengths of between 50 and 500 mm.

In some embodiments, either or both of the first group of fluid flow channels and the second group of fluid flow channels are contained within a monoblock. When both the first group of fluid flow channels and the second group of fluid flow channels are contained within a monoblock, they may be separated by channel walls formed of a material that the monoblock is formed of.

In some embodiments, the channel walls are porous and selectively permeable. In some embodiments, the selectively permeable channel walls of the fluid flow channel walls may be impregnated with one or more high boiling point liquids, which may include ionic liquids, hydrocarbons, and amines.

Consistent with some disclosed embodiments, an apparatus comprising a monoblock having a plurality of fluid flow channels, including a first group of fluid flow channels, which is independent from a second group of fluid flow channels, is disclosed. In some embodiments, the first group of fluid flow channels is structurally distinct from the second group of fluid flow channels. For example, in one embodiment, the second group of fluid flow channels (FC2) is capped at one end, whereas the first group of fluid flow channels (FC1) is open at both ends.

The monoblock may be configured to maintain a pressure differential between the first group of fluid flow channels and the second groups of fluid flow channels. In some embodiments, the plurality of fluid flow channels may be separated by channel walls, which are porous, selectively permeable, and formed of a material that the monoblock is formed of.

In some embodiments, the channel walls are impregnated with a high boiling point liquid by a process comprising the steps of: (a) selecting the high boiling point liquid with a boiling point of at least 100° C.; (b) selecting a carrier liquid with a boiling point lower than the selected high boiling point liquid; (c) mixing the high boiling point liquid with the carrier to create a solution with a concentration of high boiling point liquid of about between 15% and about 20%; and (d) impregnating the solution into at least a subset of the plurality of fluid flow channels using a process to effectuate at least partial pore infusion.

In some embodiments, the channel walls are impregnated with one or more high boiling point liquids, and at least a portion of the first group of fluid flow channels are adjacent to at least a portion of the second group of fluid flow channels. Non-limiting examples of the one or more high boiling point liquids include ionic liquids, hydrocarbons, and amines. In some embodiments, these high boiling point liquids preferentially absorb one or more components of a process fluid stream introduced to the first group of fluid flow channels. In addition, or in the alternative, the high boiling point liquids are configured to deactivate at least a subset of viruses, germs, mold spores, or other biological contaminants from a process fluid stream introduced to the first group of fluid flow channels (FC1).

In one embodiment, the one or more high boiling point liquids comprises one or more ionic liquids adapted to preferentially absorb one of more fluid selected from the group consisting of: CO2, oxygen, water vapor, CO, SOx, and NOx.

In some embodiments, the monoblock is configured to maintain a pressure differential between the first and second groups of fluid flow channels. For example, in an embodiment, the first group of fluid flow channels is under a higher pressure than the second group of fluid flow channels. In an embodiment, the first group of fluid flow channels is under a positive gauge pressure and the second group of fluid flow channels is under a negative gauge pressure.

In some embodiments, the two or more independent groups of fluid flow channels extend through a length of the porous monoblock. They may also have a surface area to volume ratio of between about 20 in\hat{ }2/in\hat{ }3 and about 200 in\hat{ }2/in\hat{ }3. The porous and selectively permeable walls of the two or more independent groups of fluid flow channels may have a pore volume of between about 0.10 mL/g and about 1.0 mL/g.

In some embodiments, the apparatus further includes a header that is attached to one or both ends of the monoblock that has at least one inlet, a transition element, a plurality of routing channels, and at least one outlet. The header's first inlet may be configured to receive a first bulk flow of fluid. In an embodiment, the at least one outlet comprises a plurality of openings that are fluidly connected to a plurality of fluid flow channels in the monoblock. In one embodiment, the transition element is configured to divide the first bulk flow of fluid into a multi-channel flow of fluid; a plurality of first routing channels are configured to route the first multi-channel flow of fluid to a first outlet; the first outlet comprising a plurality of openings that are fluidly connected to the first group of fluid flow channels and not fluidly connected to the second group of fluid flow channels; and the at least one header is attached to at least one end of the monoblock.

In some embodiments, the first group of fluid flow channels (FC1) that is independent of the second group of fluid flow channels (FC2) are arranged in a repeating pattern, such as a checkerboard pattern, offset checkerboard pattern, honeycomb pattern, quatrefoil pattern, rectangle pattern, diamond pattern, triangle pattern, triangle and diamond pattern, and triangle and rectangle pattern.

In some embodiments, the apparatus further comprises a device configured to sequester an exhaust fluid stream from the second group of fluid flow channels. In some embodiments, the monoblock may have a separation efficiency of above 50%, above 75%, or even above 98%.

Consistent with some disclosed embodiments a method of reducing the concentration of components of a fluid stream is disclosed. The method includes introducing a process fluid stream comprising a mixture of fluid species into a device having: (a) at least one header having: at least one inlet, a transition element, a plurality of routing channels, and at least one outlet; a first inlet is configured to receive a first bulk flow of fluid; the at least one outlet comprises a plurality of openings that are fluidly connected to a plurality of fluid flow channels; the transition element is configured to divide the first bulk flow of fluid into a multi-channel flow of fluid; a plurality of first routing channels are configured to route the first multi-channel flow of fluid to a first outlet; and the first outlet comprising a plurality of openings that are fluidly connected to a first group of fluid flow channels. The method also includes having a monoblock, as described above.

In one embodiment, the method of reducing the concentration of components of a fluid stream includes using a header that has a second inlet comprising a plurality of openings that are fluidly connected to the second group of fluid flow channels. The second inlet is configured to receive a second multi-channel flow of fluid from the second group of fluid flow channels. In one embodiment, a plurality of second routing channels being configured to route the second multi-channel flow of fluid from the second inlet to the transition element. The transition element is configured to merge the second multi-channel flow of fluid into a second bulk flow of fluid. A second outlet is configured to exhaust the second bulk flow of fluid from said header. The first group of fluid flow channels is structurally distinct from the second group of fluid flow channels, and the first routing channels are structurally distinct from and not in fluid communication with the second routing channels.

In some embodiments, the header may be configured to maintain a pressure differential between the first group of fluid flow channels and the second group of fluid flow channels.

In some embodiments, the header has a plenum structure with a second inlet that is fluidly connected to a second group of fluid flow channels on the device attached to the header. The second inlet of the plenum is configured to receive a second multi-channel flow of fluid from the second group of fluid flow channels. The plenum is configured to merge the second multi-channel flow of fluid to form a second bulk flow of fluid; a second outlet being configured to exhaust the second bulk flow of fluid; and the first group of fluid flow channels being structurally distinct from the second group of fluid flow channels.

Consistent with some disclosed embodiments a method of manufacturing a header is disclosed. In some embodiments, the method may comprise one or more of the following steps: (a) providing a monoblock formed of a porous material; the monoblock having a plurality of fluid flow channels formed therein; the plurality of fluid flow channels being separated by channel walls; the channel walls being porous and selectively permeable; (b) providing a negative mold comprising fingers adapted to register in a subset of the plurality of fluid flow channels; (c) inserting the fingers of the negative mold into the subset of the plurality of fluid flow channels; (d) introducing a liquid polymeric material into the negative mold; (e) allowing said liquid polymeric material to cure; and (f) removing the negative mold to establish a header.

In the disclosed method, the fingers of the negative mold may be inserted into at least a subset of the channels of the monoblock before the monoblock is hardened to facilitate alignment.

In some embodiments, the liquid polymer that is introduced into the negative mold at least partially enters the pores of the porous monoblock, such that when the negative mold is removed, the formed header is attached to the porous monoblock by virtue of the polymer having been inserted into the porous monoblock's pores.

In some embodiments, the negative mold is made from a material selected from a group consisting of metal, wax, polymer, and dissolving polymer.

### DETAILED DESCRIPTION OF THE DRAWINGS

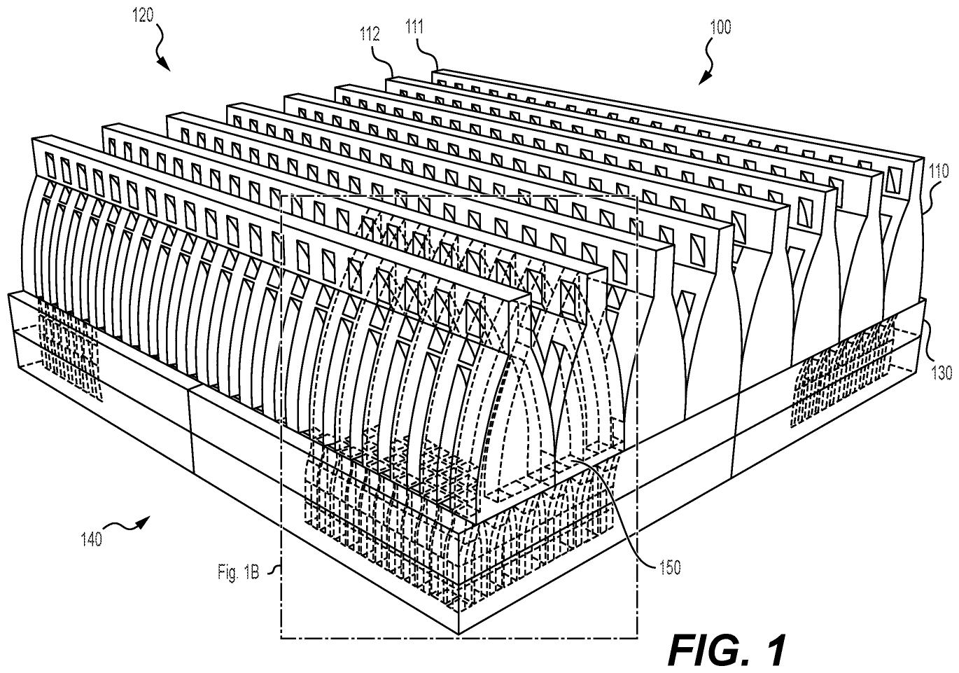

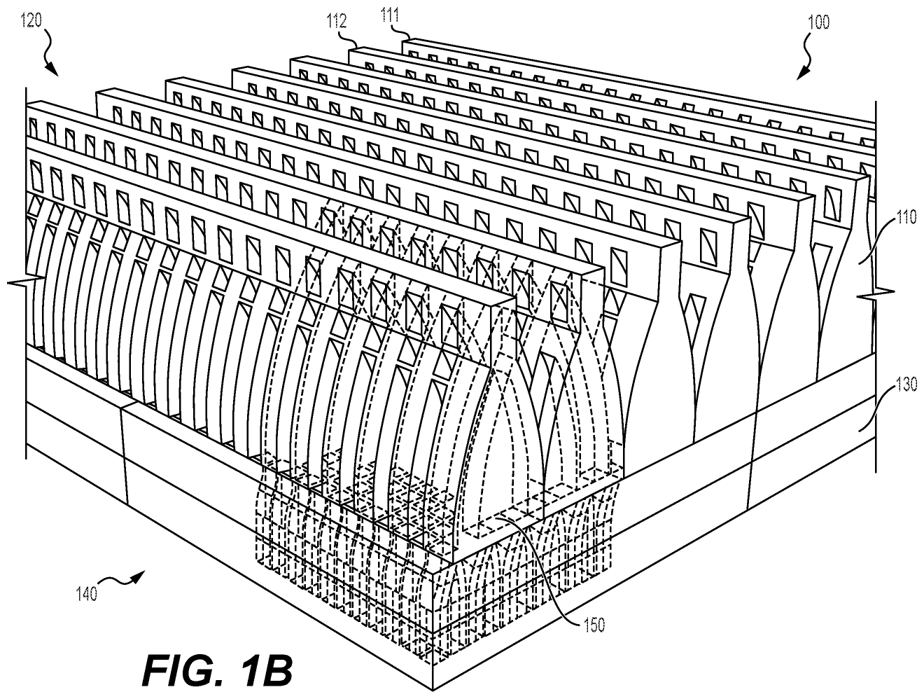

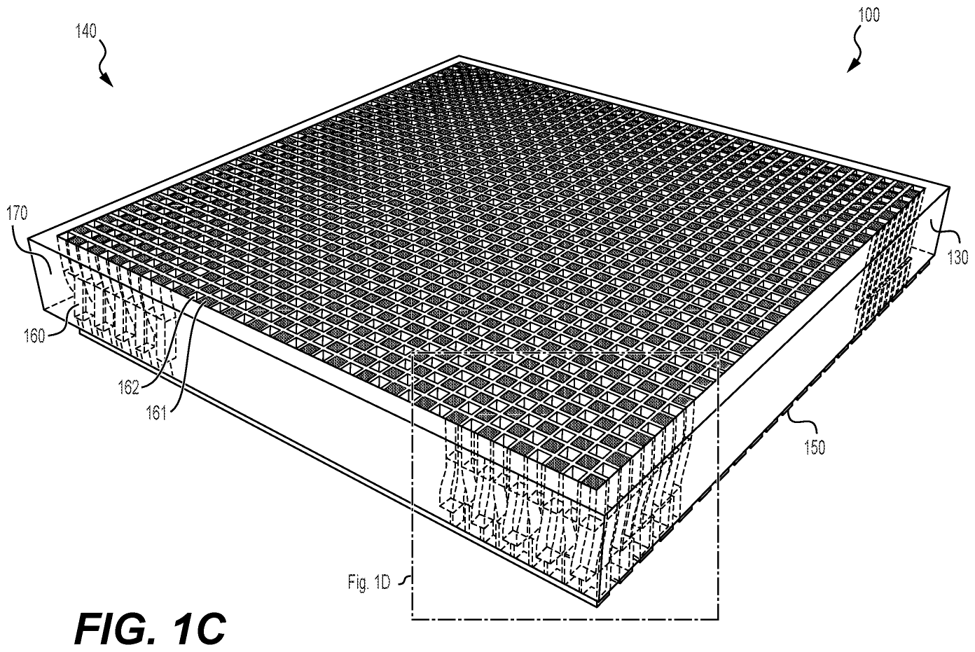

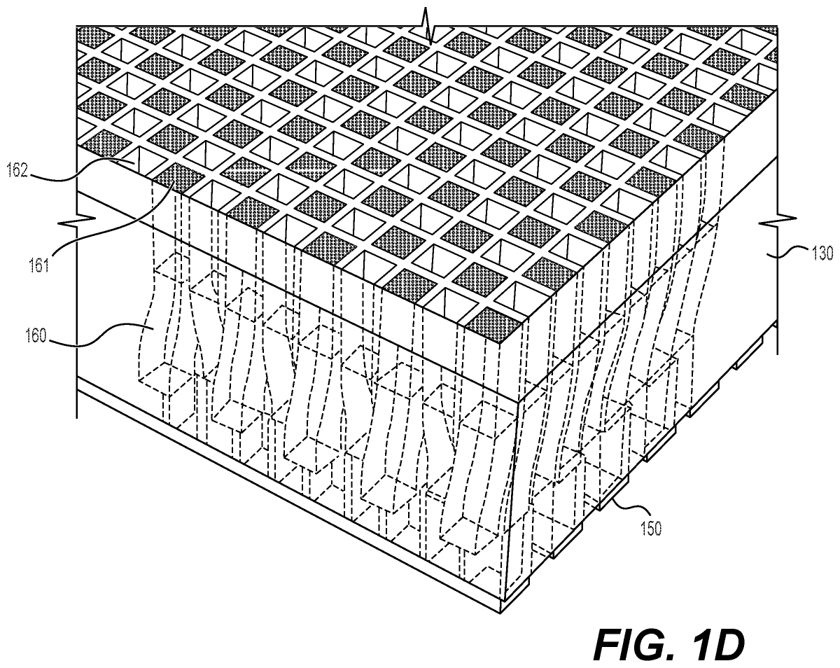

FIG. 1A is a perspective view of the header structure 100 consistent with some disclosed embodiments. The header 100 includes a plurality of partitioners 110 on side A 120 of the header. In this embodiment, the partitioners 110 are arranged to connect to a linear fitting. A partitioner 110 may be a process stream partitioner 111, which divides a bulk flow of a process fluid stream at side A 120 of the header to a multi-channel flow of fluid at a transition point 150. Or a partitioner 110 may be an exhaust stream partitioner 112, which merges a multi-channel flow of fluid of an exhaust fluid stream at a transition point 150 to a bulk flow at side A 120 of the header, for removal from the system. The partitioners 110 connect to the channelizer 130 at transition point 150. The channelizer 130 routes the multi-channel flow of fluid from a transition point 150 into or out of the relevant group of fluid flow channels of the monoblock at side B 140 of the header 100.

Therefore, a process fluid stream enters the system through a process stream partitioner 111, divides to a multi-channel flow of process fluid at a transition point 150, enters the channelizer 130, and gets routed to the first group of fluid flow channels in the monoblock. Similarly, the exhaust fluid stream exits the monoblock through the second group of fluid flow channels, enters the channelizer 130, which routes the multi-channel flow of exhaust fluid to the transition point 150 and into an exhaust stream partitioner 112, which merges the multi-channel flow of exhaust fluid into a bulk flow of exhaust fluid to be removed from the system at side A 120.

FIG. 1B is a detailed view of a portion of the header structure 100 depicted in FIG. 1A, particularly focusing on the channelizer 130 routing the multi-channel flow of fluid at a transition point 150 into or out of the relevant fluid flow channels of the monoblock at side B 140 of the header 100.

FIG. 1C is a detailed view of the channelizer 130 on side B 140 of the header structure 100 depicted in FIG. 1A. Side B 140 of the channelizer 130 connects to the monoblock. The arrangement of routing channels 160 correspond with the arrangement of channels formed in the monoblock. As seen from the side view 170 of the channelizer 130, the routing channels 160 are non-linear and route the multi-channel flow of fluid between a transition point 150 and the relevant fluid flow channels in the monoblock. A routing channel 160 may be a first routing channel 161, which routes the multi-channel flow of process fluid from a transition point 150 to a first group of fluid flow channels in the monoblock. Or a routing channel 160 may be a second routing channel 162, which routes an exhaust fluid stream from the second group of fluid flow channels in the monoblock to a transition point 150 for removal from the system. In this embodiment, the first and second routing channels, 161 and 162, respectively, form a checkerboard pattern.

FIG. 1D is a detailed view of the channelizer depicted in FIG. 1C. The arrangement of routing channels 160 correspond with the arrangement of fluid flow channels formed in the monoblock. The routing channels 160 are non-linear and route the multi-channel flow of fluid between a transition point 150 and the relevant fluid flow channels in the monoblock. A routing channel 160 may be a first routing channel 161, which routes the multi-channel flow of process fluid from a transition point 150 to a first group of fluid flow channels in the monoblock. Or a routing channel 160 may be a second routing channel 162, which routes an exhaust fluid stream from the second group of fluid flow channels in the monoblock to a transition point 150 for removal from the system. In this embodiment, the first and second routing channels, 161 and 162, respectively, form a checkerboard pattern.

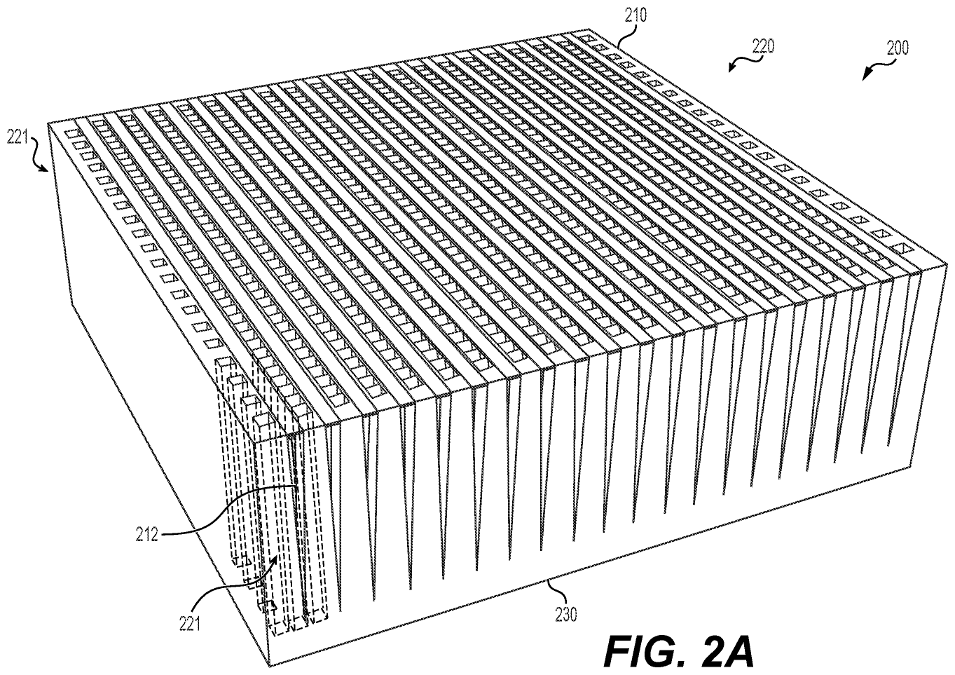

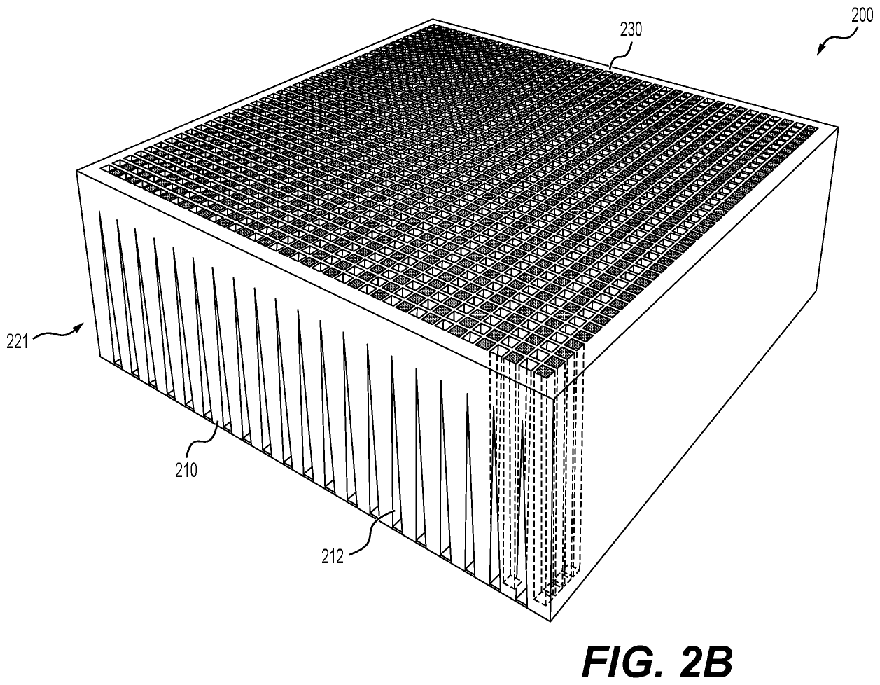

FIG. 2A is a perspective view of the header structure 200 consistent with some disclosed embodiments. The header 200 includes a plurality of partitioners 210 on side A1 220 and on sides A2 221 of the header. A partitioner 210 may be a process stream partitioner 211, which divides a bulk flow of a process fluid stream at side A1 220 of the header to a multi-channel flow of process fluid at a transition point 240. Or a partitioner 210 may be an exhaust stream partitioner 212, which merges a multi-channel flow of exhaust fluid from a transition point 240 to a bulk flow of exhaust fluid at sides A2 221 of the header, for removal from the system. In this embodiment, the pressure drop between the bulk flow and multi-channel flow of fluid is reduced. The partitioners 210 connect to the channelizer 130, as depicted in FIG. 1C above, at a transition point 230.

FIG. 2B is an alternate perspective view of the header structure 200 depicted in FIG. 2A. The figure depicts side A2 221 and exhaust stream partitioners 212. The transition point 240 depicts alternating rows of a multi-channel flow of exhaust fluid and a multi-channel flow of process fluid. The partitioners 210 connect to the channelizer 130, as depicted in FIG. 1C above, at the transition point 230.

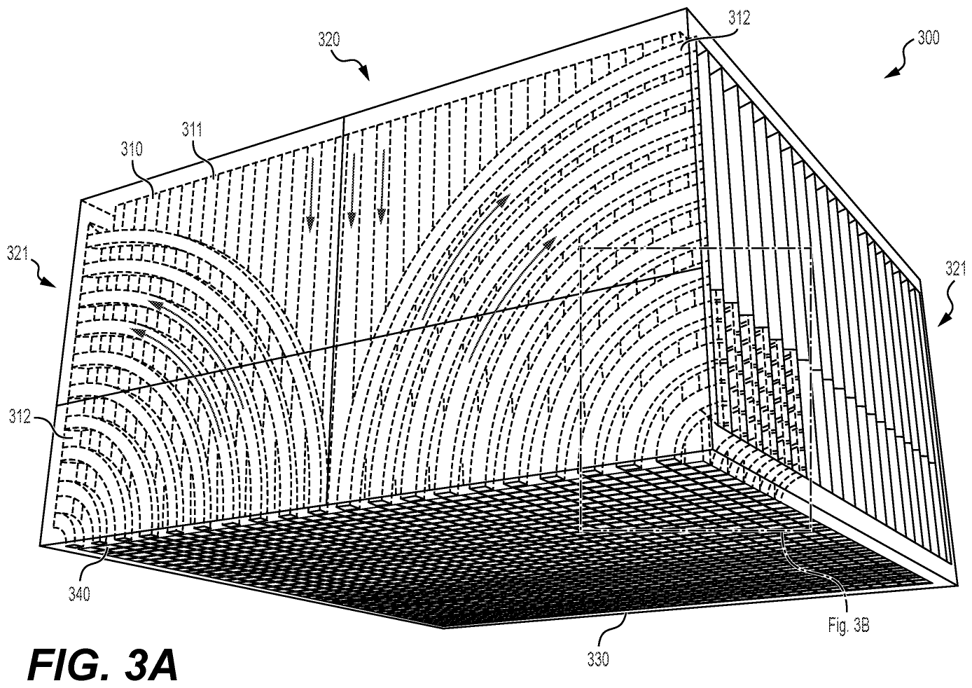

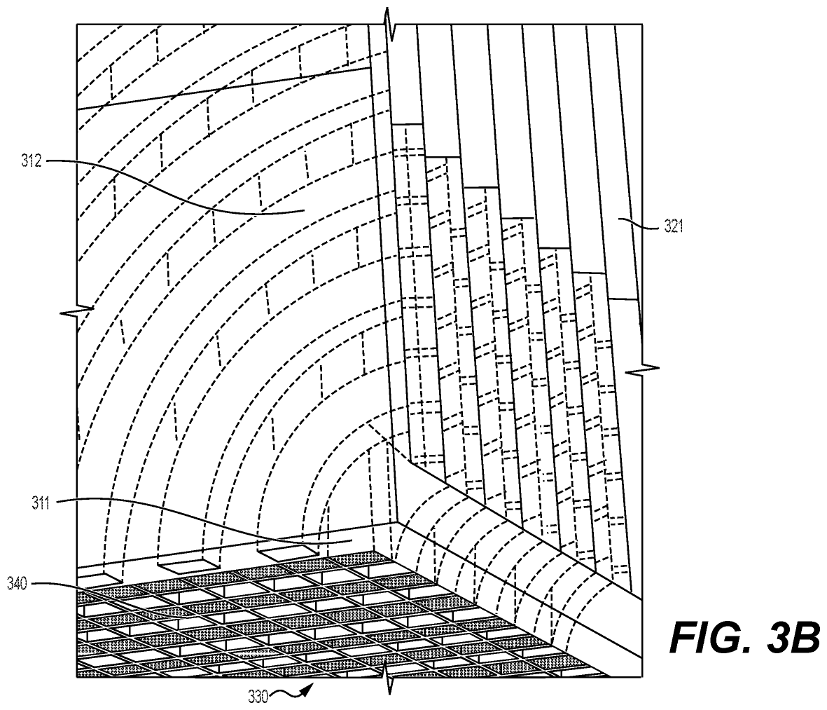

FIG. 3A is a perspective view of the header structure 300 consistent with some disclosed embodiments. The header 300 includes a plurality of partitioners 310 on side A1 320 and on sides A2 321 of the header. In this embodiment, the partitioners 310 are arranged to connect to a rectangular fitting on either or all of sides A1 320 and A2 321. A partitioner 310 may be a process stream partitioner 311, which divides a bulk flow of a process fluid stream at side A1 320 of the header to a multi-channel flow of fluid at a transition point 340. Or a partitioner 310 may be an exhaust stream partitioner 312, which merges a multi-channel flow of exhaust fluid from a transition point 340 to a bulk flow of exhaust fluid at sides A2 321 of the header, for removal from the system. The partitioners 310 connect to the channelizer 130, as depicted in FIG. 1C above, at a transition point 330.

FIG. 3B is a detailed view of the header in FIG. 3A. The figure depicts side A2 321 and exhaust stream partitioners 312. The transition point 340 depicts the exhaust stream partitioners 312 creating a multi-channel flow of exhaust fluid in a row and the process stream partitioners 311 creating a multi-channel flow of process fluid in an adjacent row. The partitioners 310 connect to the channelizer 130, as depicted in FIG. 1C above, at the transition point 340.

The transition point 340 depicts the exhaust stream partitioners 312 and the process stream partitioners 311 creating alternating rows of multi-channel flows of exhaust and process fluids. The partitioners 310 connect to the channelizer 130, as depicted in FIG. 1C above, at a transition point 340.

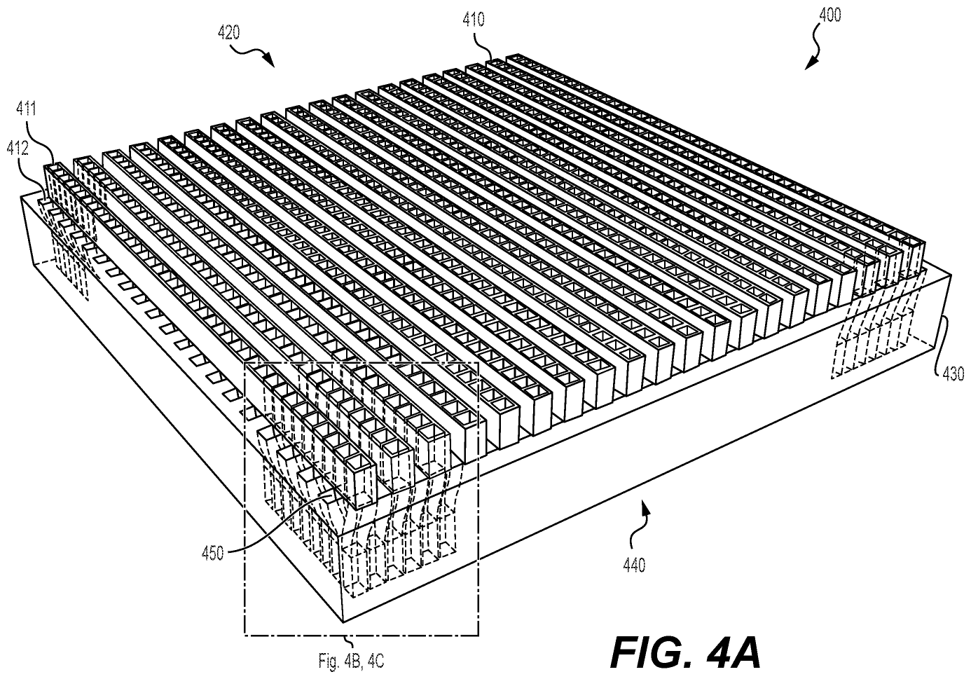

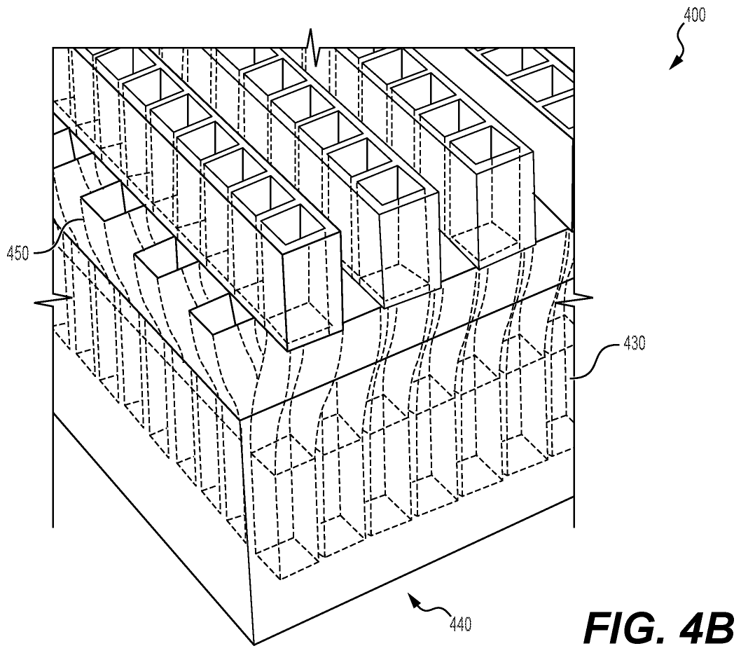

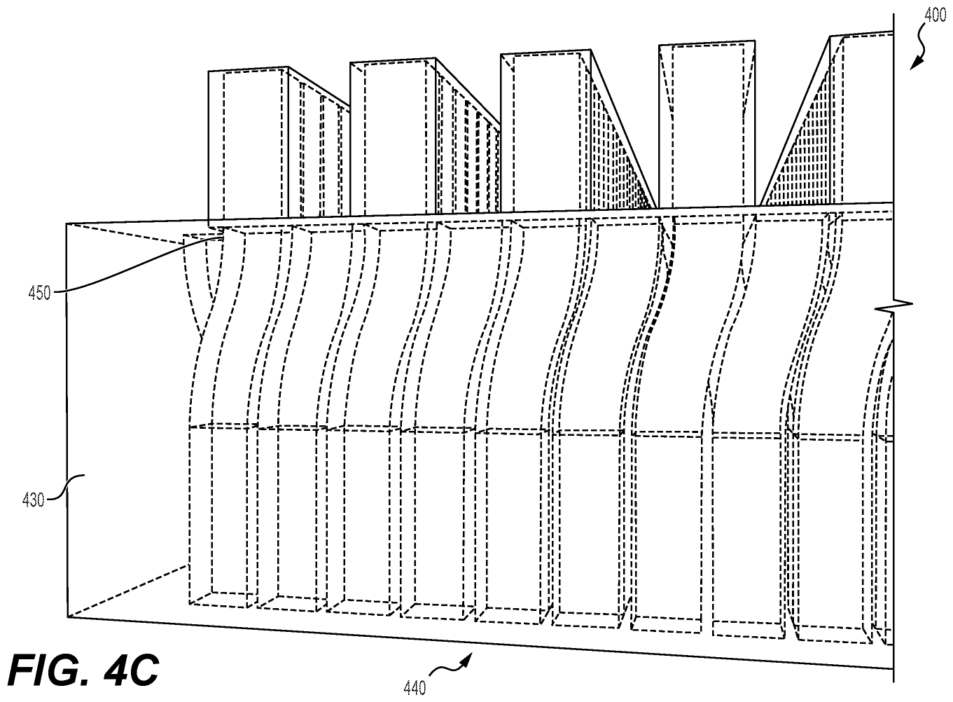

FIG. 4A is a perspective view of the header structure 400 consistent with some disclosed embodiments. The header 400 includes a plurality of partitioners 410 on side A 420 of the header. In this embodiment, the partitioners 410 are arranged to connect to a linear fitting. A partitioner 410 may be a process stream partitioner 411, which divides a bulk flow of a process fluid stream at side A 420 of the header into a multi-channel flow of fluid at a transition point 450. Or a partitioner 410 may be an exhaust stream partitioner 412, which merges a multi-channel flow of fluid from a transition point 450 to a bulk flow at side A 420 of the header, for removal from the system. The partitioners 410 connect to the channelizer 430 at a transition point 450. The channelizer 430 routes the multi-channel flow of fluid between a transition point 450 and the relevant fluid flow channels in the monoblock, at side B 440 of the header 400.

Therefore, a process fluid stream enters the system through a process stream partitioner 411, divides into a multi-channel flow of process fluid at a transition point 450, enters the channelizer 430, and gets routed into the first group of fluid flow channels in the monoblock. Similarly, the exhaust fluid stream exits the monoblock through the second group of fluid flow channels, enters the channelizer 430, which routes the multi-channel flow of exhaust fluid to a transition point 450 and into an exhaust stream partitioner 412, which transitions the multi-channel flow of exhaust fluid into a bulk flow of exhaust fluid to be removed from the system at side A 420 of the header.

FIGS. 4B and 4C are detailed views of a portion of the header structure 400 depicted in FIG. 4A, particularly focusing on the channelizer 430 routing the multi-channel flow of fluid between a transition point 450 and the relevant fluid flow channels of the monoblock.

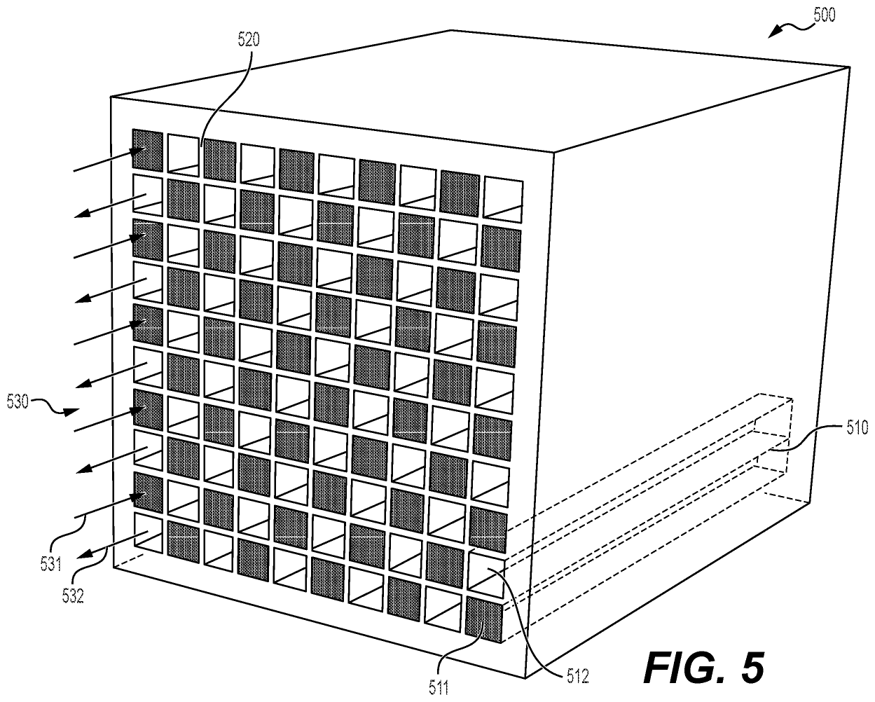

FIG. 5 is a perspective view of the monoblock structure 500 depicting the flow of fluid streams 530, consistent with some disclosed embodiments. This view of the monoblock structure 500 shows a plurality of fluid flow channels 510 formed within the monoblock 500. The fluid flow channels 510 may or may not extend fully through the monoblock structure 500. The fluid flow channels 510 include a first group of fluid flow channels 511 and a second group of fluid flow channels 512, which are separated by channel walls 520. The channel walls 520 are made of substantially the same material as the monoblock structure 500, but are impregnated with a high boiling point liquid. The figure shows fluid streams 530, including a process fluid stream 531 and an exhaust fluid stream 532.

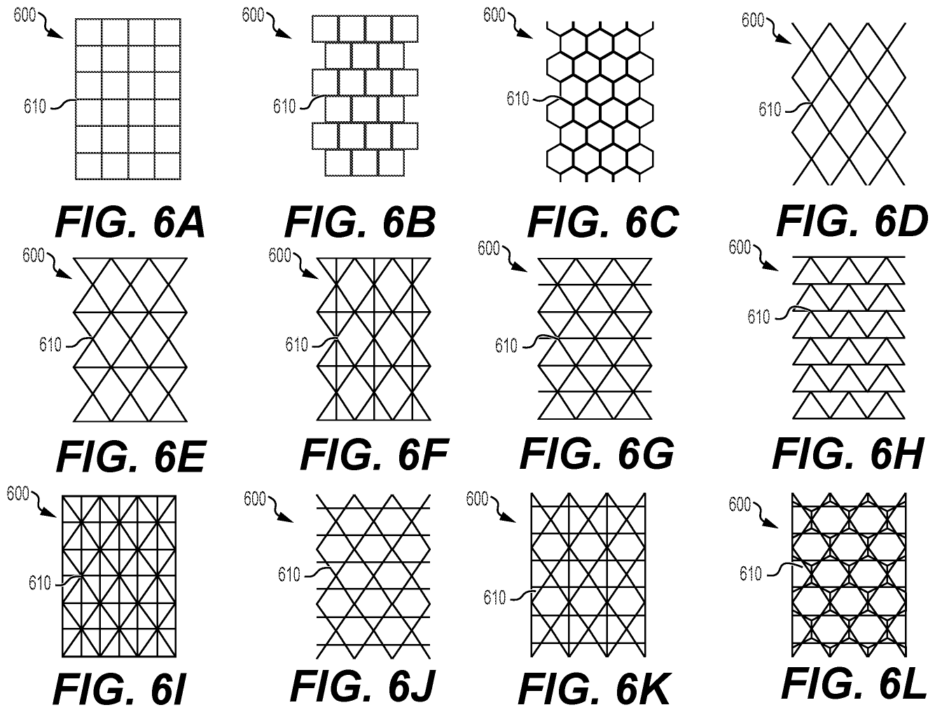

FIG. 6A to 6L are exemplary arrangements of the fluid flow channels formed within a monoblock, consistent with some disclosed embodiments. FIG. 6A shows that the channels 600 are arranged in a checkerboard pattern and each channel is separated by a channel wall 610. FIG. 6B shows the channels 600 arranged in an off-set checkerboard pattern and each channel is separated by a channel wall 610. FIG. 6C shows the channels 600 are arranged in a honeycomb pattern and each channel is separated by a channel wall 610. FIG. 6D shows the channels 600 arranged in a diamond pattern and each channel is separated by a channel wall 610. FIG. 6E shows the channels 600 arranged in a diamond and triangle pattern and each channel is separated by a channel wall 610. FIG. 6F shows the channels 600 arranged in an alternate diamond and triangle pattern and each channel is separated by a channel wall 610. FIG. 6G shows the channels 600 arranged in an equilateral triangle pattern and each channel is separated by a channel wall 610. FIG. 6H shows the channels 600 arranged in an offset triangle pattern and each channel is separated by a channel wall 610. FIG. 6I shows the channels 600 arranged in a right triangle pattern and each channel is separated by a channel wall 610. FIG. 6J shows the channels 600 arranged in a triangle and pentagon pattern and each channel is separated by a channel wall 610. FIG. 6K shows the channels 600 arranged in an alternate triangle and pentagon pattern and each channel is separated by a channel wall 610. FIG. 6L shows the channels 600 arranged in a triangle and hexagon pattern and each channel is separated by a channel wall 610.

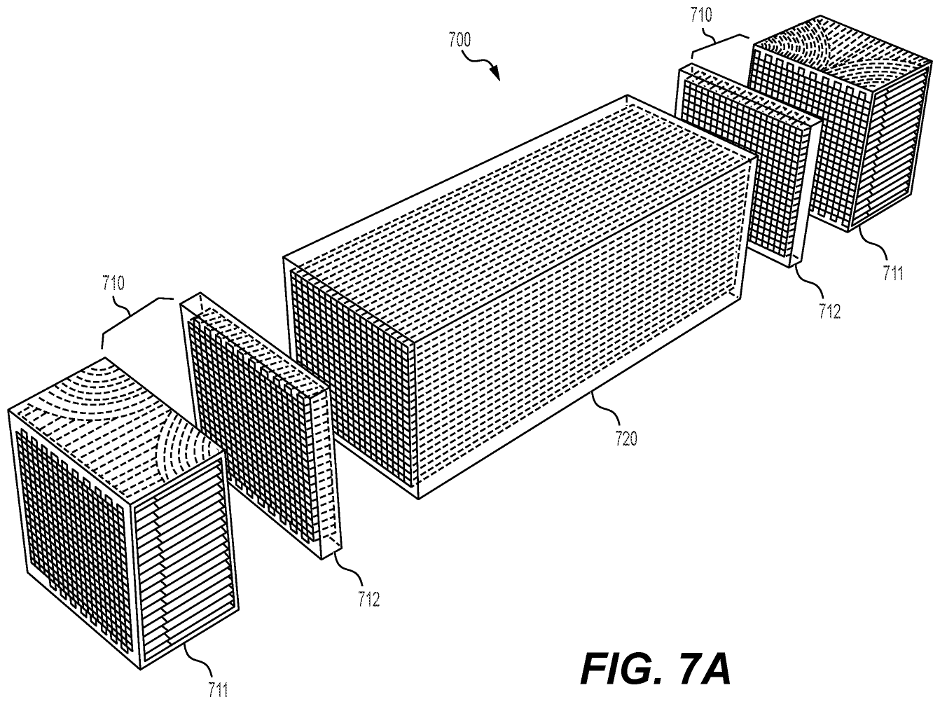

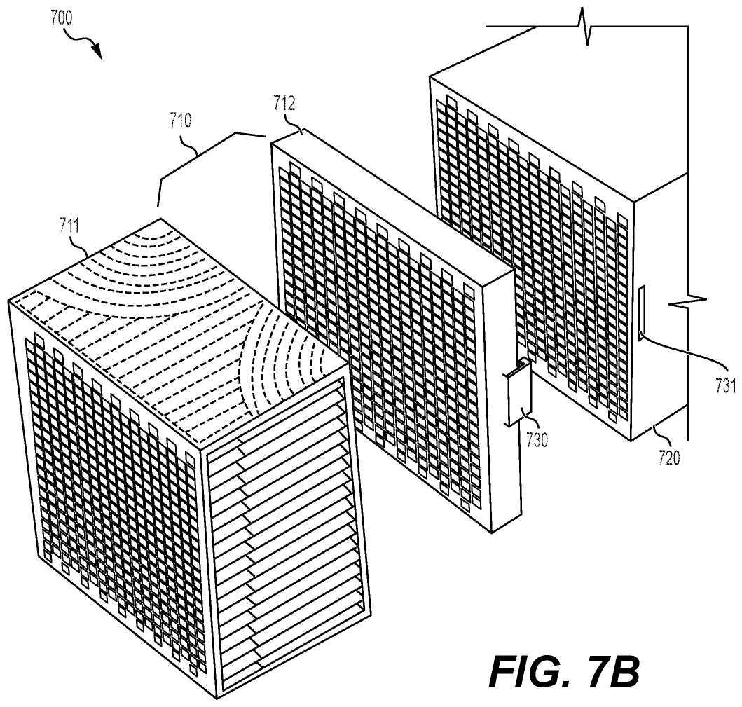

FIG. 7A is an exploded perspective view of the fluid separation apparatus including a header 710, comprising a partitioner 711 and a channelizer 712, and a monoblock 720.

FIG. 7B is a detailed view of the fluid separation apparatus of FIG. 7A. It shows that the partitioner 711 is connected to the channelizer 712 to form a header 710. The header 710 may be either removably or permanently connected to the monoblock 720. In this exemplary embodiment, the header 710 is removably connected to the monoblock 720, as shown by clip 730 on the header 710 and the indent 731 to removably receive the clip 730, or any other mechanism or substance to removably connect.

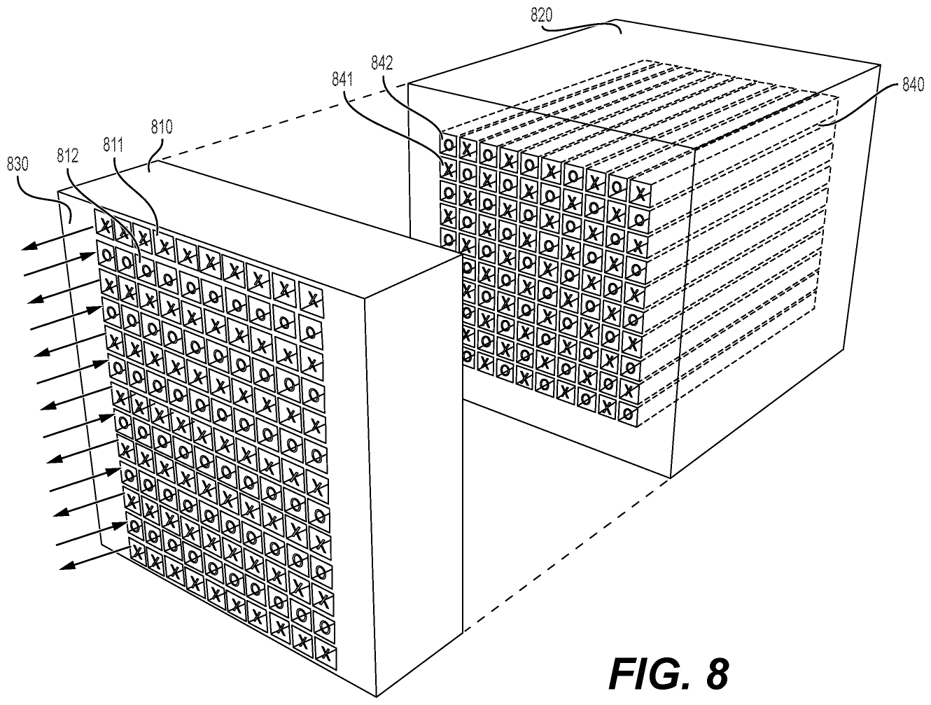

FIG. 8 depicts the channelizer 810 routing fluid streams between a transition point 830 and the relevant fluid flow channels 840 of the monoblock 820 consistent with some disclosed embodiments. The face of the channelizer 810 depicts a multi-channel flow of fluid at a transition point 830, comprising a multi-channel flow of process fluid 811 and a multi-channel flow of exhaust fluid 812, each of which are in alternating rows. The channelizer 810 routes the multi-channel flow of process fluid 811 and a multi-channel flow of exhaust fluid 812, between the transition point 830 and the relevant fluid flow channels 840 of the monoblock 820. In this exemplary embodiment, the rows of the multi-channel flow of process fluid 811 get routed into the first group of fluid flow channels 841 in the monoblock 820, which are in a checkerboard arrangement. And the rows of the multi-channel flow of exhaust fluid 812 get routed out of the second group of fluid flow channels 842 in the monoblock 820, which are also in a checkerboard arrangement.

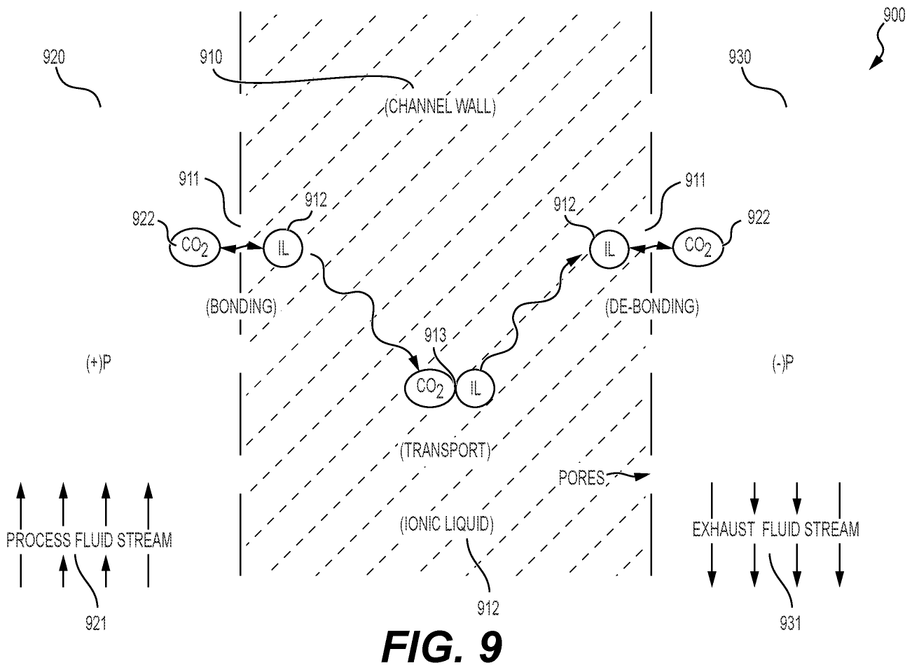

FIG. 9 is the mechanism of target fluid flow 900 through the channel walls 910 of a monoblock consistent with some disclosed embodiments. The figure includes a first group of fluid flow channels 920, through which a process fluid stream 921 flows, and a second group of fluid flow channels 930, through which an exhaust fluid stream 931 flows. The first group of fluid flow channels 920 is at a higher pressure compared to the second group of fluid flow channels 930.

Channel walls 910 separate the first group of fluid flow channels 920 and the second group of fluid flow channels 930. The channel walls 910 have pores 911 on both sides of the channel wall 910 that create permeability through the channel walls 910. The channel walls 910 are impregnated with a high boiling point liquid, which in this embodiment, is an ionic liquid 912. In other embodiments, the high boiling point liquid may be only partially impregnated in the channel walls, or impregnated in only the first half of the channel walls starting from the portion of the channel walls closest to the first group of fluid flow channels.

A process fluid stream 921 comprising a mixture of fluid species, including a target fluid species 922, enters the monoblock in the first group of fluid flow channels 920. As the process fluid stream 921 flows through the first group of fluid flow channels 920, one or more target fluid species 922, for example CO2 in this exemplary embodiment, becomes attracted to the ionic liquid 912 impregnated in the channel walls 910. The ionic liquid 912 will then absorb to at least a portion of the target fluid species 922 through the pores 911 in the channel walls 910.

The target fluid species—ionic liquid compound 913—diffuses across the channel wall 910 due to the pressure differential across the channel walls 910. When the compound 913 reaches the opposite side of the channel wall 910, the relatively lower pressure will cause the target fluid species 922 to desorb from the ionic liquid 912. Once the target fluid species 922 is desorbed, it will enter the second group of fluid flow channels 930 through a pore 911 in the channel wall 910. The target fluid species 922 then exits the monoblock as an exhaust fluid stream 931 via the second group of fluid flow channels 930. In this exemplary embodiment, the process fluid stream 921 and the exhaust fluid stream 931 flow counter-current to one another.

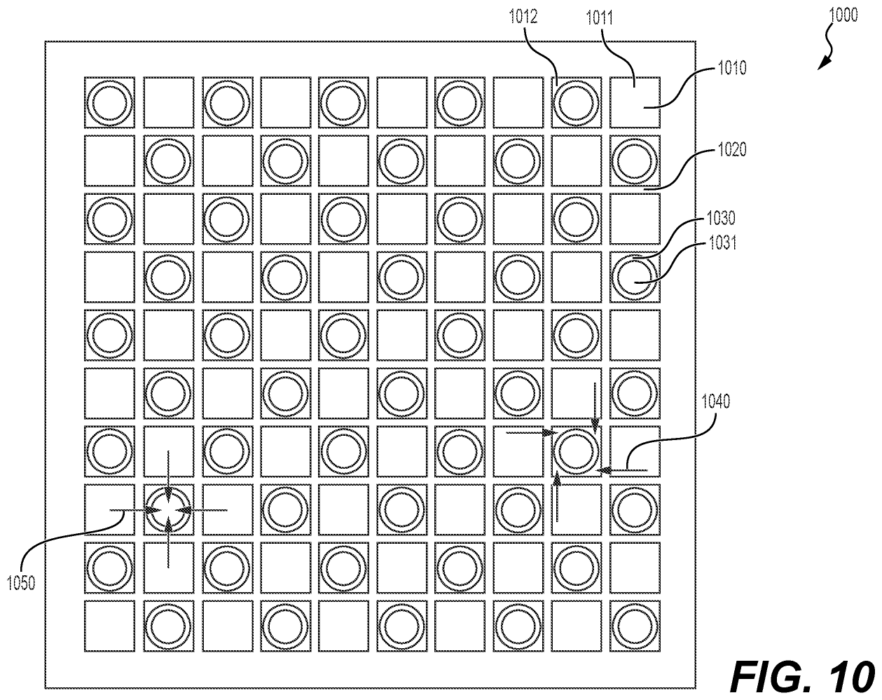

FIG. 10 is a two-dimensional front view of the monoblock structure 1000 consistent with some disclosed embodiments. This front view of the monoblock structure 1000 shows a plurality of fluid flow channels 1010 formed within the monoblock 1000. The fluid flow channels 1010 include a first group of fluid flow channels 1011 and a second group of fluid flow channels 1012, which are separated by thermally conductive channel walls 1020. The channel walls 1020 are made of substantially the same material as the monoblock structure 1000, but are impregnated with a high boiling point liquid.

As depicted in FIG. 9, a target fluid species 1040 transfers from the first group of fluid flow channels 1011 to the second group of fluid flow channels 1012 through the channel wall 1020, which is impregnated with a high boiling point liquid.

This embodiment can also heat or cool the process fluid stream contained in the first group of fluid flow channels. Tubes 1030 are inserted into the second group of fluid flow channels 1012. The tubes 1030 are made of a thermally conductive material and carry either heating or cooling fluid 1031. The tubes 1030 make physical contact with the channel walls 1020 on the interior of the second group of fluid flow channels 1012. If the tubes 1030 contain a cooling fluid 1031, heat 1050 will flow from the process fluid stream to the channel walls 1020, through the thermally conductive channel walls 1020, through the thermally conductive tube 1030, and into the cooling fluid 1031.

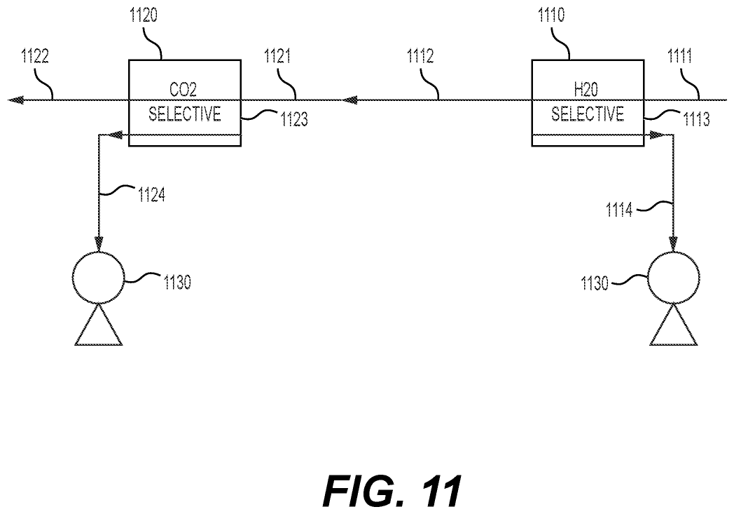

FIG. 11 is a block diagram of two monoblocks 1110 and 1120 arranged in series consistent with some disclosed embodiments. In this exemplary embodiment, there is a first monoblock 1110, which is impregnated with a first high boiling point liquid 1113 with an affinity for water vapor, and a second monoblock 1120, which is impregnated with a second first high boiling point liquid 1123 with an affinity for CO2. The first monoblock 1110 receives a first process fluid stream 1111 and separates at least a portion of the water vapor into the first exhaust fluid stream 1114. The first conditioned process fluid stream 1112 exits the first monoblock 1110 and serves as a second process fluid stream 1121. The second monoblock 1120 receives the second process fluid stream 1121 and separates at least a portion of the CO₂ into the second exhaust fluid stream 1124. The second conditioned process fluid stream 1122 then exits the second monoblock 1120. In this exemplary embodiment, vacuum pumps 1130 generated negative pressure to extract the first exhaust fluid stream 1114 and second exhaust fluid stream 1124 from the first monoblock 1110 and the second monoblock 1120, respectively. In this exemplary embodiment, the first process fluid stream 1111 may be outdoor air and the second conditioned process fluid stream 1122 may be directed into a building. This arrangement may be preferable when building occupants prefer air with low humidity and low CO2 content.

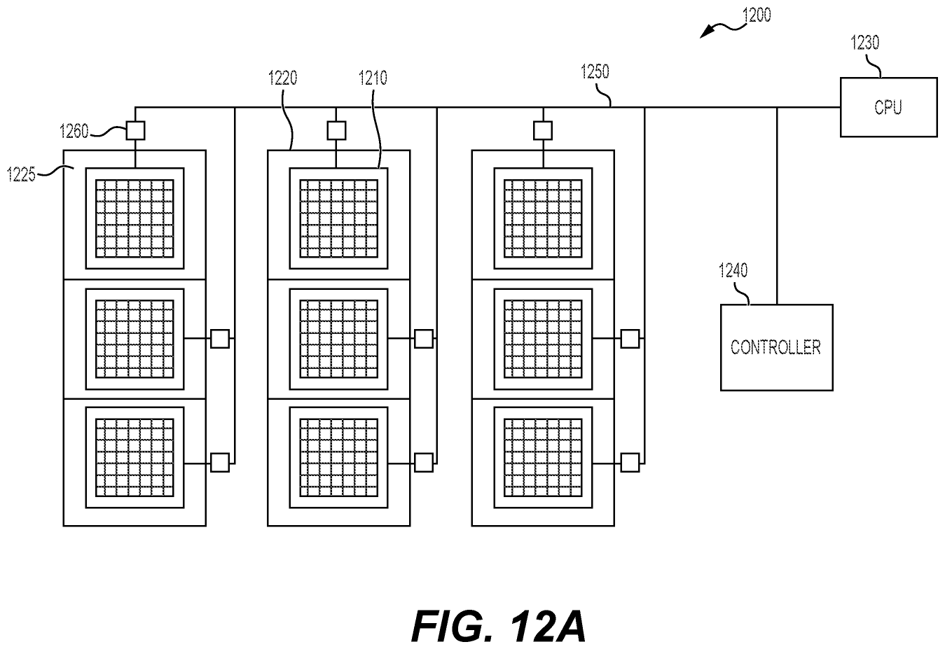

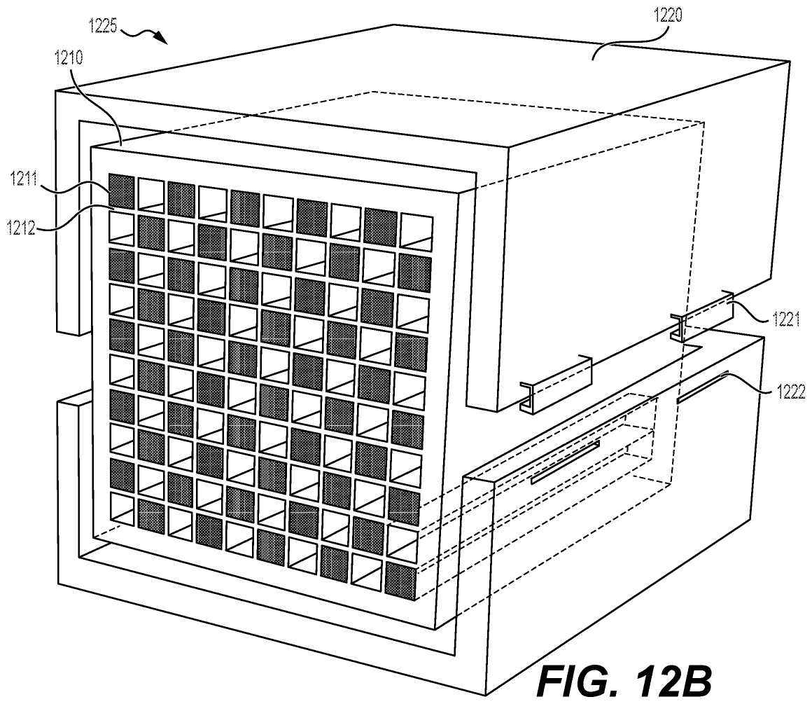

FIG. 12A is a block diagram of a fluid conditioning unit 1200 (“FCU”). In an application where the continuous conditioning of a process fluid stream is desirable, an FCU may be provided, comprising an array of monoblocks 1210 to condition a process fluid stream in parallel, series, or a combination thereof using pipes or tubes 1250. Conditioning a process fluid stream in parallel allows one or more monoblocks to be switched out during operation of the conditioning process in the event that one or more monoblocks 1210 fails during operation or requires routine maintenance. A housing 1220 may be provided to enclose and seal at least one monoblock 1210 and header, if needed. In this arrangement, the combination of a monoblock 1210 and a housing 1220 is a line replaceable unit (“LRU”) 1225 and can be replaced individually in the event of monoblock 1210 failure or for routine maintenance.

In this embodiment, each housing 1220 may incorporate sensors 1260 to monitor the performance and operation of the monoblock 1210 incorporated within that housing 1220. The sensors for each of the housings may communicate with a central processing unit (“CPU”) 1230 to send an alert when a housing 1220 needs replacement. The CPU 1230 also may connect to a central controller 1240, which may divert the process fluid stream away from LRUs 1225 that need replacement.

FIG. 12B is a perspective view of a line replaceable unit (“LRU”) 1225, consistent with some disclosed embodiments. In this exemplary embodiment, the LRU 1225 comprises a housing 1220 enclosing and sealing a monoblock 1210 and a header, if needed. The housing 1220 may be opened to provide for access to the monoblock 1210 housed within. To facilitate opening, the housing may have a clip 1221 and a divot 1222 to accept the clip 1221, or any other mechanism or substance to removably connect. The housing 1220 may also have connecting mechanisms such as snaps, clips, latches to removably connect one LRU 1225 to one or more adjacent LRUs 1225 and/or to a structural scaffolding.

The foregoing description, including dimensions and numbers of certain elements, is presented for purposes of illustration. It is not exhaustive and is not limited to precise forms or embodiments disclosed. Modifications and adaptations of the embodiments will be apparent from consideration of the specification and practice of the disclosed embodiments. While certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application. Such examples are to be construed as nonexclusive. Further, the steps of the disclosed methods can be modified in any manner, including reordering steps and/or inserting or deleting steps.

The features and advantages of this disclosure are apparent from this detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Throughout this application, various embodiments of the present disclosure may be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numeric values within that range. For example, description of a range such as from 1 to 6 should be considered to include subranges such as from 1 to 3, from 1 to 4, from 1 to 5, from 2 to 4, from 2 to 6, from 3 to 6, and so forth, as well as individual numbers within that range, for example, 1, 2, 3, 4, 5, and 6. This applies regardless of the breadth of the range.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A header configured to divide a bulk flow of fluid into a multi-channel flow of fluid, comprising:
at least one inlet, a transition element, a plurality of routing channels, and at least one outlet;
a first inlet is configured to receive a first bulk flow of fluid;
the at least one outlet comprises a plurality of openings that are fluidly connected to a plurality of fluid flow channels on a device attached to the header;
the transition element is configured to divide the first bulk flow of fluid into a first multi-channel flow of fluid;
a plurality of first routing channels is configured to route the first multi-channel flow of fluid to a first outlet; and
a first outlet comprises a plurality of openings that are fluidly connected to a first group of fluid flow channels.

2. The header of claim 1, further comprising:
a second inlet comprising a plurality of openings that are fluidly connected to a second group of fluid flow channels on the device attached to the header;
the second inlet being configured to receive a second multi-channel flow of fluid from the second group of fluid flow channels;
a plurality of second routing channels are configured to route the second multi-channel flow of fluid from the second inlet to the transition element;
the transition element being further configured to merge the second multi-channel flow of fluid into a second bulk flow of fluid;
a second outlet configured to exhaust the second bulk flow of fluid from said header;
the first group of fluid flow channels is structurally distinct from the second group of fluid flow channels; and
the first routing channels are structurally distinct from and not in fluid communication with the second routing channels.

3. The header of claim 1, further comprising:
The header being configured to maintain a pressure differential between the first group of fluid flow channels and a second group of fluid flow channels.

4. The header of claim 1, further comprising:
a plenum structure;
a second inlet that is fluidly connected to a second group of fluid flow channels on the device attached to the header;
the second inlet being configured to receive a second multi-channel flow of fluid from the second group of fluid flow channels;
the plenum structure is configured to merge the second multi-channel flow of fluid to form a second bulk flow of fluid;
a second outlet is configured to exhaust the second bulk flow of fluid; and
the first group of fluid flow channels is structurally distinct from the second group of fluid flow channels.

5. The header of claim 4, further comprising:
said plenum structure being selected from a group consisting of a housing, a manifold, ductwork, a chamber, an air distribution box, and a cavity.

6. The header of claim 1, further comprising:
the header being at least partially made of a material selected from the group consisting of polymer, dissolvable polymer, composite material, wax, and metal.

7. The header of claim 1, further comprising:
the first group of fluid flow channels and a second groups of fluid flow channels comprising between 2 fluid flow channels and 1600 fluid flow channels.

8. The header of claim 1, further comprising:
the first group of fluid flow channels and a second groups of fluid flow channels having a size of between about 200 channels/in\hat{ }2 and about 1200 channels/in\hat{ }2.

9. The header of claim 1, further comprising:
the first group of fluid flow channels and a second group of fluid flow channels having lengths of between about 50 mm and about 500 mm.

10. The header of claim 1, further comprising:
the first group of fluid flow channels being contained within a monoblock.

11. The header of claim 1, further comprising:
both the first group of fluid flow channels and a second group of fluid flow channels being contained within a monoblock; and
wherein the first group of fluid flow channels and the second group of fluid flow channels are separated by channel walls formed of a material that the monoblock is formed of.

12. The header of claim 11, further comprising:
said channel walls being porous and selectively permeable.

13. The header of claim 12, further comprising:
said channel walls being impregnated with one or more high boiling point liquids selected from the group consisting of ionic liquids, hydrocarbons, and amines.

14. An apparatus, comprising:
a monoblock having a plurality of fluid flow channels, including a first group of fluid flow channels, which is independent from a second group of fluid flow channels;
said plurality of fluid flow channels are separated by channel walls;
said channel walls being porous, selectively permeable, and formed of a material that the monoblock is formed of;
the channel walls being impregnated with one or more high boiling point liquids; and
at least a portion of the first group of fluid flow channels being adjacent to at least a portion of the second group of fluid flow channels.

15. The apparatus of claim 14, further comprising:
the first group of fluid flow channels being structurally distinct from the second group of fluid flow channels.

16. The apparatus of claim 14, further comprising:
the monoblock being configured to maintain a pressure differential between the first group of fluid flow channels and the second groups of fluid flow channels.

17. The apparatus of claim 14, further comprising:
the channel walls being impregnated with a high boiling point liquid by a process comprising the steps of:
(a) selecting the high boiling point liquid with a boiling point of at least about 100° C.;
(b) selecting a carrier liquid with a boiling point lower than the selected high boiling point liquid;
(c) mixing the high boiling point liquid with the carrier to create a solution with a concentration of high boiling point liquid of about between 15% and about 20%; and
(d) impregnating the solution into at least a subset of the plurality of fluid flow channels using a process to effectuate at least partial pore infusion.

18. The apparatus of claim 14, further comprising:
the one or more high boiling point liquids being selected from the group consisting of ionic liquids, hydrocarbons, and amines.

19. The apparatus of claim 14, further comprising:
the one or more high boiling point liquids being configured to preferentially absorb one or more components of a process fluid stream introduced to the first group of fluid flow channels.

20. The apparatus of claim 14, further comprising:
the one or more high boiling point liquids are configured to deactivate at least a subset of viruses, dust, mites, bacteria, germs, mold spores, or other biological contaminants from a process fluid stream introduced to the first group of fluid flow channels.

21. The apparatus of claim 14, further comprising:
the plurality of fluid flow channels having a surface area to volume ratio of between about 20 in\hat{ }2/in\hat{ }3 and about 200 in\hat{ }2/in\hat{ }3.

22. The apparatus of claim 14, further comprising:
the channel walls having a pore volume of between about 0.1 mL/g and about 1.0 mL/g.

23. The apparatus of claim 14, further comprising:
the first group of fluid flow channels being under a higher pressure than the second group of fluid flow channels.

24. The apparatus of claim 14, further comprising:
the first group of fluid flow channels being under a positive gauge pressure and the second group of fluid flow channels being under a negative gauge pressure.

25. The apparatus of claim 14, further comprising:
the second group of fluid flow channels is restricted at one end.

26. The apparatus of claim 14, further comprising:
at least one header having at least one inlet, a transition element, a plurality of routing channels, and at least one outlet;
a first inlet is configured to receive a first bulk flow of fluid;
the at least one outlet comprises a plurality of openings that are fluidly connected to a plurality of fluid flow channels in the monoblock;
the transition element is configured to divide the first bulk flow of fluid into a first multi-channel flow of fluid;
a plurality of first routing channels are configured to route the first multi-channel flow of fluid to a first outlet;
the first outlet comprising a plurality of openings that are fluidly connected to the first group of fluid flow channels and not fluidly connected to the second group of fluid flow channels; and
the at least one header being attached to at least one end of the monoblock.

27. The apparatus of claim 14, further comprising:
the first group of fluid flow channels and the second group of fluid flow channels being arranged in a repeating pattern.

28. The apparatus of claim 27, further comprising:
the repeating pattern being selected from the group consisting of a checkerboard pattern, offset checkerboard pattern, honeycomb pattern, quatrefoil pattern, rectangle pattern, diamond pattern, triangle pattern, triangle and diamond pattern, and triangle and rectangle pattern.

29. The apparatus of claim 14, further comprising:
the one or more high boiling point liquids comprises one or more ionic liquids configured to preferentially absorb one of more fluid selected from the group consisting of: CO2, oxygen, water vapor, CO, SOx, NOx, helium, and formaldehyde.

30. The apparatus of claim 14, further comprising:
a device configured to sequester an exhaust fluid stream;
the device being fluidly connected to the second group of fluid flow channels.

31. The apparatus of claim 14, further comprising:
the apparatus having a separation efficiency of at least about 50%.

32. A method of reducing a concentration of one or more components of a process fluid stream, comprising:
introducing a process fluid stream comprising a mixture of fluid species into a device having:
(a) at least one header having:
at least one inlet, a transition element, a plurality of routing channels, and at least one outlet;
a first inlet is configured to receive a first bulk flow of fluid;
the at least one outlet comprises a plurality of openings that are fluidly connected to a plurality of fluid flow channels;
the transition element is configured to divide the first bulk flow of fluid into a first multi-channel flow of fluid;
a plurality of first routing channels are configured to route the first multi-channel flow of fluid to a first outlet;
the first outlet comprising a plurality of openings that are fluidly connected to a first group of fluid flow channels;
(b) a monoblock having a plurality of fluid flow channels including the first group of fluid flow channels and a second group of fluid flow channels;
said plurality of fluid flow channels are separated by channel walls;
the channel walls being porous, selectively permeable, and formed of a material that the monoblock is formed of;
the channel walls are impregnated with one or more high boiling point liquids; and
at least a portion of the first group of fluid flow channels being adjacent to at least a portion of the second group of fluid flow channels.

33. The method of claim 32, further comprising:
The header having:
a second inlet comprising a plurality of openings that are fluidly connected to the second group of fluid flow channels;
the second inlet being configured to receive a second multi-channel flow of fluid from the second group of fluid flow channels;
a plurality of second routing channels being configured to route the second multi-channel flow of fluid from the second inlet to the transition element;
the transition element being configured to merge the second multi-channel flow of fluid into a second bulk flow of fluid;
a second outlet being configured to exhaust the second bulk flow of fluid from said header;
the first group of fluid flow channels being structurally distinct from the second group of fluid flow channels; and
the first routing channels are structurally distinct from and not in fluid communication with the second routing channels.

34. The method of claim 32, further comprising:
the header being configured to maintain a pressure differential between the first group of fluid flow channels and the second group of fluid flow channels.

35. The method of claim 32, further comprising:
the header having:
a plenum structure;
a second inlet that is fluidly connected to a second group of fluid flow channels on the device attached to the header;
wherein the second inlet is configured to receive a second multi-channel flow of fluid from the second group of fluid flow channels;
wherein the plenum structure is configured to merge the second multi-channel flow of fluid to form a second bulk flow of fluid;
a second outlet being configured to exhaust the second bulk flow of fluid; and
the first group of fluid flow channels being structurally distinct from the second group of fluid flow channels.

36. The method of claim 35, further comprising:
said plenum structure being selected from a group consisting of a housing, a manifold, ductwork, a chamber, an air distribution box, and a cavity.

37. The method of claim 32, further comprising:
the header being at least partially made of a polymeric or composite material.

38. The method of claim 32, further comprising:
the plurality of fluid flow channels comprising 2-1600 fluid flow channels.

39. The method of claim 32, further comprising:
the plurality of fluid flow channels having a size of between about 200 channels/in\hat{ }2 and about 1200 channels/in\hat{ }2.

40. The method of claim 32, further comprising:
the plurality of fluid flow channels having a length of between about 50 mm and about 500 mm.

41. The method of claim 32, further comprising:
the first group of fluid flow channels being structurally distinct from the second group of fluid flow channels.

42. The method of claim 32, further comprising:
the monoblock being configured to maintain a pressure differential between the first group of fluid flow channels and the second group of fluid flow channels.

43. The method of claim 32, further comprising:
the one or more high boiling point liquids being selected from the group consisting of ionic liquids, hydrocarbons, and amines.

44. The method of claim 32, further comprising:
the one or more high boiling point liquids being configured to preferentially absorb one or more components of a process fluid stream introduced to the first group of fluid flow channels.

45. The method of claim 32, further comprising:
the one or more high boiling point liquids being configured to deactivate at least a subset of viruses, dust, mites, bacteria, germs, mold spores, or other biological contaminants from a process fluid stream introduced to the first group of fluid flow channels.

46. The method of claim 32, further comprising:
the plurality of fluid flow channels extending through a length of the monoblock.

47. The method of claim 32, further comprising:
the plurality of fluid flow channels having a surface area to volume ratio of between about 20 in\hat{ }2/in\hat{ }3 and about 200 in\hat{ }2/in\hat{ }3.

48. The method of claim 32, further comprising:
the channel walls having a pore volume of between about 0.1 mL/g and about 1.0 mL/g.

49. The method of claim 32, further comprising:
the first group of fluid flow channels being under a higher pressure than the second group of fluid flow channels.

50. The method of claim 32, further comprising:
the first group of fluid flow channels being under a positive gauge pressure and the second group of fluid flow channels being under a negative gauge pressure.

51. The method of claim 32, further comprising:
the second group of fluid flow channels being restricted at one end.

52. The method of claim 32, further comprising:
the first group of fluid flow channels and the second group of fluid flow channels being arranged in a repeating pattern.

53. The method of claim 52, further comprising:
the repeating pattern being selected from the group consisting of a checkerboard pattern, offset checkerboard pattern, honeycomb pattern, quatrefoil pattern, rectangle pattern, diamond pattern, triangle pattern, triangle and diamond pattern, and triangle and rectangle pattern.

54. The method of claim 32, further comprising:
the one or more high boiling point liquids comprising one or more ionic liquids configured to preferentially absorb one of more fluid species selected from the group consisting of CO2, oxygen, water vapor, CO, SOx, NOx, helium, and formaldehyde.

55. The method of claim 32, further comprising:
a device configured to sequester an exhaust fluid stream;
the device being fluidly connected to the second group of fluid flow channels.

56. The method of claim 32, further comprising:
the method having a separation efficiency of at least about 50%.

57. A method of manufacturing a header, comprising the steps of:
(a) providing a monoblock formed of a porous material;
the monoblock having a plurality of fluid flow channels formed therein;
the plurality of fluid flow channels being separated by channel walls;
the channel walls being porous and selectively permeable;
(b) providing a negative mold comprising fingers adapted to register in a subset of the plurality of fluid flow channels;
(c) inserting the fingers of the negative mold into the subset of the plurality of fluid flow channels;
(d) introducing into the negative mold a liquid material selected from the group consisting of polymer, dissolvable polymer, wax, and metal;
(e) allowing said liquid polymeric material to cure; and
(f) removing the negative mold to establish a header.

58. The method of claim 57, further comprising:
the fingers of the negative mold being inserted into the subset of the plurality of fluid flow channels before the monoblock is hardened to facilitate alignment.

59. The method of claim 57, further comprising:
the liquid material that is introduced into the negative mold entering a plurality of pores in the porous material of the monoblock;
the liquid material being allowed to at least partially cure; and
the negative mold being removed to create a header attached to the porous monoblock by virtue of the liquid material having entered into the plurality of pores in the porous material of the monoblock.
